(19) United States
(12) Patent Application Publication
(10) Pub. No.: US 2002/0025307 A1
(43) Pub. Date: Feb. 28, 2002

(54) Bone sialoprotein based toxic gene therapy for the treatment of calcified tumors and tissues

(76) Inventors: Kenneth S. Koeneman, Charlottesville, VA (US); Leland W.K. Chung, Lovingston, VA (US)

Correspondence Address: PENNIE & EDMONDS LLP, 1667 K STREET NW, SUITE 1000, WASHINGTON, DC, 20006

(21) Appl. No.: 09/884,098

(22) Filed: Jun. 20, 2001

Related U.S. Application Data
(63) Continuation-in-part of application No. PCT/US99/30642, filed on Dec. 22, 1999.
(60) Provisional application No. 60/113,200, filed on Dec. 22, 1998.

(51) Int. Cl. A61K 48/00; A61K 38/17; A61K 31/56; A61K 31/505; A61K 31/522

(52) U.S. Cl. 424/93.21; 514/44; 514/8; 514/171; 514/263.38; 514/256

(57) ABSTRACT

The present invention relates to promoters, enhancers and other regulatory elements that direct expression within tumor and tissue cells with calcification potential. In particular, it relates to compositions comprising nucleotide sequences from the 5′ regulatory region, and transcriptionally active fragments thereof, that control expression of a bone sialoprotein (“BSP”). Specifically provided are expression vectors, host cells and transgenic animals wherein a BSP regulatory region is capable of controlling expression of a heterologous coding sequence, over-expressing an endogenous BSP coding sequence or an inhibitor of a pathological process or knocking out expression of a specific gene believed to be important for a calcification-related disease in tumor and tissue cells with calcification potential. The invention also relates to methods for using said vectors, cells and animals for screening candidate molecules for agonists and antagonists of disorders involving tumor and tissue cells with calcification potential. The present invention further relates to compositions and methods for modulating expression of compounds within tumor and tissue cells with calcification potential. Methods for using molecules and compounds identified by screening assays for therapeutic treatments also are provided. The invention further relates to methods of treating tumors and other diseases and disorders involving tumor and tissue cells with calcification potential.

This application is a continuation-in-part of PCT Application No. PCT/US99/30642 filed Dec. 22, 1999, which claims priority under 35 U.S.C. §119(e) to U.S. provisional patent application no. 60/113,200 filed Dec. 22, 1998, each of which is hereby incorporated by reference in its entirety.

### 1 INTRODUCTION

The present invention relates to promoters, enhancers and other regulatory elements that direct expression within tumor and tissue cells with calcification potential. In particular, it relates to compositions comprising nucleotide sequences from the 5′ regulatory region, and transcriptionally active fragments thereof, that control expression of a bone sialoprotein (“BSP”). Specifically provided are expression vectors, host cells and transgenic animals wherein a BSP regulatory region is capable of controlling expression of a heterologous coding sequence, over-expressing an endogenous BSP coding sequence or an inhibitor of a pathological process or knocking out expression of a specific gene believed to be important for a calcification-related disease in tumor and tissue cells with calcification potential. The invention also relates to methods for using said vectors, cells and animals for screening candidate molecules for agonists and antagonists of disorders involving tumor and tissue cells with calcification potential.

The present invention further relates to compositions and methods for modulating expression of compounds within tumor and tissue cells with calcification potential. The invention further relates to screening compounds that modulate expression within tumor and tissue cells with calcification potential. Methods for using molecules and compounds identified by the screening assays for therapeutic treatments also are provided. The invention further relates to methods of treating tumors and other diseases and disorders involving tumor and tissue cells with calcification potential.

The present invention further relates to targeted therapy using recombinant vectors and particularly adenovirus vectors. The invention specifically relates to replication-conditional adenovirus vectors and methods for using them. Such adenovirus vectors are able to selectively replicate in a tissue-specific and tumor-restrictive manner to provide a therapeutic benefit from the presence of the adenovirus vector per se and/or from heterologous gene products expressed from the vector. In particular, the transcriptional regulatory sequences utilized with the adenoviral vectors of the present invention are capable of selectively driving expression of an adenovirus gene essential for replication in a tissue-specific and tumor-restrictive manner. Thus, due to the tissue specificity of the transcriptional regulatory sequences used with the viral vectors, the viral vectors of the present invention are effective therapeutic agents not only when administered via direct application, such as by injection into the target tissue, but also when administered systemically to the body via intravenous administration, oral administration or the like, because gene expression will be limited and localized to specific cell types.

### 2 BACKGROUND OF THE INVENTION

2.1 Gene Therapy

Somatic cell gene therapy is a strategy in which a nucleic acid, typically in the form of DNA, is administered to alter the genetic repertoire of target cells for therapeutic purposes. Although research in experimental gene therapy is a relatively young field, major advances have been made during the last decade. (Arai, Y., et al., 1997, Orthopaedic Research Society, 22:341). The potential of somatic cell gene therapy to treat human diseases has caught the imagination of numerous scientists, mainly because of two recent technologic advancements. Firstly, there are now numerous viral and non-viral gene therapy vectors that can efficiently transfer and express genes in experimental animals in vivo Secondly, increasing support for the human genome project will allow for the identity and sequence of the estimated 80,000 genes comprising the human genome in the very near future.

Gene therapy was originally conceived of as a specific gene replacement therapy for correction of heritable defects to deliver functionally active therapeutic genes into targeted cells. Initial efforts toward somatic gene therapy relied on indirect means of introducing genes into tissues, called ex vivo gene therapy, e.g., target cells are removed from the body, transfected or infected with vectors carrying recombinant genes and reimplanted into the body (“autologous cell transfer”). A variety of transfection techniques are currently available and used to transfer DNA in vitro into cells; including calcium phosphate-DNA precipitation, DEAE-Dextran transfection, electroporation, liposome mediated DNA transfer or transduction with recombinant viral vectors. Such ex vivo treatment protocols have been proposed to transfer DNA into a variety of different cell types including epithelial cells (U.S. Pat. No. 4,868,116; Morgan and Mulligan WO87/00201; Morgan et al, 1987, Science 237:1476-1479; Morgan and Mulligan, U.S. Pat. No. 4,980,286), endothelial cells (WO89/05345), hepatocytes (WO89/07136; Wolff et al., 1987, Proc. Natl. Acad. Sci. USA 84:3344-3348; Ledley et al., 1987 Proc. Natl. Acad. Sci. 84:5335-5339; Wilson and Mulligan, WO89/07136; Wilson et al., 1990, Proc. Natl. Acad. Sci. 87:8437-8441), fibroblasts (Palmer et al., 1987, Proc. Natl. Acad. Sci. USA 84:10551059; Anson et al, 1987, Mol. Biol. Med. 4:11-20; Rosenberg et al., 1988, Science 242:1575-1578; Naughton & Naughton, U.S. Pat. No. 4,963,489), lymphocytes (Anderson et al., U.S. Pat. No. 5,399,346; Blaese, R. M. et al., 1995, Science 270:475-480) and hematopoietic stem cells (Lim, B. et al. 1989, Proc. Natl. Acad. Sci. USA 86:8892-8896; Anderson et al., U.S. Pat. No. 5,399,346).

Direct in vivo gene transfer recently has been attempted with formulations of DNA trapped in liposomes (Ledley et al., 1987, J. Pediatrics 110:1), in proteoliposomes that contain viral envelope receptor proteins (Nicolau et al., 1983, Proc. Natl. Acad. Sci. U.S.A. 80:1068) and DNA coupled to a polylysine-glycoprotein carrier complex. In addition, “gene guns” have been used for gene delivery into cells (Australian Patent No. 9068389). It even has been speculated that naked DNA, or DNA associated with liposomes, can be formulated in liquid carrier solutions for injection into interstitial spaces for transfer of DNA into cells (Felgner, WO90/11092).

Numerous clinical trials utilizing gene therapy techniques are underway for such diverse diseases as cystic fibrosis and cancer. The promise of this therapeutic approach for dramatically improving the practice of medicine has been supported widely, although there still are many hurdles that need to be passed before this technology can be used successfully in the clinical setting.

Perhaps, one of the greatest problems associated with currently devised gene therapies, whether ex vivo or in vivo, is the inability to control expression of a target gene and to limit expression of the target gene to the cell type or types needed to achieve a beneficial therapeutic effect.

The concept of delivery and expression of therapeutic toxic genes to tumor cells through the use of tissue-specific promoters has been well recognized. This approach decreases the toxic effect of therapeutic genes on neighboring normal cells when vector (virus, liposome, etc.) gene delivery results in the infection of the normal cells as well as the cancerous cells. Examples include the uses of α-fetoprotein promoter to target hepatoma cells (Koryama, et al., 1991, Cell Struct. Punct., 16:503-510), the carcinoembryonic antigen (CEA) promoter for gastric carcinoma (Tanaka, et al., 1996, Cancer Research, 46:1341-1345), the tyrosinase promoter to kill melanoma cells (Vile, et al., 1994, Cancer Research, 54:6228-6234), the bone morphogenic protein promoter for brain to target glioma cells (Shimizu, K., 1994, Nipson Rinsbo, 52:3053-3058), and the osteocalcin promoter to kill osteocarcinoma and prostate cancers (Ko, S. et al., 1996, Cancer Research, 56:4614-4619; Gardener, et al., 1998, Gene Therapy and Molecular Biology, 2:41-58). Molecular therapeutic strategies such as gene therapy through use of tissue and tumor-restricted promoters are being used with increasing frequency. The key components of a gene therapy approach include: i) the selection of appropriate tissue-specific or tumor-restricted promoters, which, in some instances, may be inducible by a hormone, vitamin, an antibiotic, drug or heavy metal; ii) the selection of therapeutic (or toxic) genes; iii) the appropriate vectors, such as retrovirus, adenovirus, liposomes, etc. Key to targeting the appropriate tumor tissue while sparing the normal host tissue is a promoter that can home the therapeutic genes to only those tissues which use the chosen promoter.

2.2 Tissue Specific Expression Within Tumor and Tissue Cells With Calcification Potential

The treatment of osteotropic tumors such as breast, osteosarcoma and prostate which have metastacised is a major challenge. These seemingly unrelated diseases, however, unite through a molecular analysis of the gene(s) that may be overexpressed in these forms of cancer during disease progression.

BSP is a noncollagenous bone protein in which tissue expression is limited to fully-differentiated osteoblasts in bone or other rare mineralized tissues, including tumors (Sodek J. et al., 1995, Conn. Tissue Research, 32:209-217). When evaluating human prostate cancer cells that have a propensity to metastasize to the skeleton, a surprising finding was that these cells have the ability to synthesize and secrete large amounts of non-collagenous bone matrix proteins, such as osteopontin (OPN) (Thalman GN, et al., 1997, Principles of Practice of Genitourinary Oncology, 409-416), osteocalcin (OC) (Curatolo C, et al., 1992, European Urology, 1:105-107), and BSP (Withold W., et al., 1997, Clinical Chemistry, 85-91). BSP is a 34 kilodalton protein rich in aspartic acid, glutamic acid, and glycine, with 50% of its carbohydrate rich in sialic acid. BSP is sulfated and phosphorylated (30% of serine residues), and contains a cell binding motiff sequence which is homologous to vitronectin (Oldbrg et al., 1988, J. Biol. Chem., 263:19433). BSP is involved in the nucleation front of mineralization during new bone formation, and binds hydroxyappatite tightly (Hunter et. al., 1993, PNAS, 90:8562; Chen et. al., 1992, JBMR, 7:987; Kobayashi et. al., 1996, J. Biochem., 119:475). Human BSP exists as a single copy gene on chromosome 4 (Fisher L. W. et. al., 1990, J. B. C., 265:4:2347-2351) and has 7 exons and 6 introns. BSP is distinct from other sialoproteins, such as, for example, dentin sialoprotein, osteopontin, IL-1, IL-6, TNF and other bone associated sialoproteins.

BSP is found in mature, bone-forming cells, but not in immature precursors (Bianco, et. al., 1991, Conn. Tiss. Int., 49:421; Chen et. al., 1991, Matrix, 11:133). BSP is found in trophoblastic cells of the placenta, and in cementum and dentin of teeth, but is absent in most other tissues, including unlineralized cartilage, intestine, kidney, liver, heart, and skeletal muscle (Macneil et. al., 1994, JBMR, 9:1597). In a transgenic mouse system, the activity of the BSP promoter was present at high levels in bone, but absent in kidney, liver, stomach, intestine, and spleen (Chen et. al., 1996, JBMR, 11:5:654-64), and the administration of exogenous glucocorticoid stimulated the expression of reporter gene 1.6 to 11 fold (Chen, et. al., 1996, Conn. Tiss. Res., 35:33-39). The DNA sequence of the BSP promoter is over 2000 base pairs long and contains numerous regulatory elements which include vitamin D, AP-1, glucocorticoid (GRE), hox, NFκb, TGF-β, CRE, etc. (Kim R. H., et. al., 1994, Matrix Biology. 14:31-40; Sodek J., et. al., 1996, Connective Tissue Research, 35:23-31; Kim R. H., et. al., 1996, Biochem. J., 318:219-226; Yamauchi M., et. al., 1996, Matrix Biology, 15:119-130; Kerr J. M., et. al., 1997, Calcif. Tiss. Int., 60:276282; Ogata Y., et. al., 1995, European J. Biochem., 230:183-192).

### 3 SUMMARY OF THE INVENTION

The invention disclosed herein provides a model for osteotropic-specific gene transcription. The invention is based in part on the identification of a novel therapeutic agent for treating, curing and/or ameliorating tumors with calcification potential, including, but not limited to, localized or disseminated osteosarcoma, lung, colon, melanoma, thyroid, brain, multiple myeloma, and especially including, without limitation, breast and prostate cancers. The invention specifically targets sites of metastases of the above mentioned osteotropic tumors, and where applicable, their supporting osseous stroma in the metastatic environment. In addition, the present invention also relates to therapeutic agents which may also be applicable to benign conditions, such as benign prostatic hyperplasia (BPH) or arterial sclerotic conditions where calcification occurs.

The bone sialoprotein (“BSP”) promoter represents a novel sequence which has high activity in osteotropic tumors, and can be used as a powerful tool to direct the action of a chosen therapeutic gene to these osteotropic tumors in a tumor and tissue-restricted fashion. To date, the best studied therapeutic gene is herpes simplex virus thymidine kinase (HSVTK or TK) gene. Herpes simplex virus-TK converts the pro-drug ACV (or related drug) to a phosphorylated form that is cytotoxic to dividing cells (Moolten, F. L., 1996, Cancer Research, 46:5276-5281). Critical to successfil results is the “bystander” affect, which confers cytotoxicity on neighboring non-transduced cells; effective tumor cell kill can be achieved without the delivery to and expression of suicide genes in every tumor cell in vivo. This approach has been demonstrated recently to be efficacious in causing regression of many solid tumors in animal models (Tong, X. W. et al., 1998, Anticancer Research, 18:713-718). Often, in the recent literature, the vector for delivery of the chosen tumor-specific promoter and therapeutic (toxic) genes has been recombinant adenovirus containing the selected expression cassette, or a liposomal or retroviral formulation. A number of methods of vector delivery can be implemented, including injection intralesionally, intravenously, intraosseously, or loco-regionally by perfusion. Central to effective gene therapy is the choice of a tumor-specific promoter.

Because of the poor response rate of previously treated patients with relapsed prostate cancer (or other osteotropic tumors) to conventional radiotherapy, surgery, or chemotherapy, it is important to develop new therapeutic approaches that can be applied either independently or in conjunction with current or other novel treatment modalities. The current invention provides the major advance of identifying a novel therapeutic gene that drives expression of therapeutic or toxic genes in a tumor and tissue-specific manner. More specifically, the instant invention provides, for the first time, inter alia, the identification of a novel therapeutic gene comprising the BSP promoter to direct osteotropic-specific expression, both in vitro in cultured osteotropic cells, and in vivo in transgenic animals.

The present invention provides a novel therapeutic composition comprising a BSP promoter that drives the expression of a therapeutic or toxic gene, for example herpes simplex virus thymidine kinase (TK) which is delivered by a vector, such as a recombinant adenovirus (Ad), to a variety of human tumors or benign tissues that exhibit the ability to calcify either in the primary or at metastatic sites. This is especially evident for prostate and osteosarcoma tumors, but includes any osteotropic aggressive metastatic tumor such as, for example, lung, multiple myeloma, breast, colon and brain. Non-tumor cells which have the ability to calcify and thus express BSP also are able to express high levels of the recombinant reporter or therapeutic genes of the present invention.

The present invention also provides a method of treating osteosarcoma or prostate cancer or other osteotropic tumors that are able to use the BSP promoter by the above routes of recombinant adenovirus Ad-BSP-TK administration in combination with a prodrug, most commonly acyclovir (ACV), although the BSP promoter-driven therapy is not limited to a specific vector or therapeutic gene. Indeed, conceivably, many forms of vector or toxic gene can be generated and combined with this novel BSP promoter-driven strategy to obtain a desired antitumor effect. Based upon the level and tissue-specificity of BSP promoter expression, when combined with a recombinant vector and toxic or therapeutic gene, the present invention will effectively eliminate prostate and osteosarcoma or other osteotropic tumors, including, but not limited to, lung, colon, melanoma, thyroid, brain, multiple myeloma, and breast cancers growth both in vitro, and in vivo as localized and as osseous metastatic deposits.

The present invention provides compositions and methods for screening compounds that modulate expression within osteotropic cells and tissues. In particular, it provides compositions comprising nucleotides from the human BSP promoter, and transcriptionally active fragments thereof, as well as nucleic acids that hybridize under highly stringent conditions to such nucleotides, that control the expression of an osteotropic-specific gene. Specifically provided are expression vectors comprising the BSP promoter, and transcriptionally active fragments thereof, operably associated to a heterologous reporter gene, e.g., luciferase, and host cells and transgenic animals containing such vectors. The invention also provides methods for using such vectors, cells and animals for screening candidate molecules for agonists and antagonists of osteotropic-related disorders. Methods for using molecules and compounds identified by the screening assays for therapeutic treatments also are provided.

For example, and not by way of limitation, a composition comprising a reporter gene is operatively linked to a BSP promoter. The BSP driven reporter gene is expressed as a transgene in animals. The transgenic animal, and cells derived from osteotropic cells of such transgenic animal, can be used to screen compounds for candidates useful for modulating osteotropic-related disorders. Without being bound by any particular theory, such compounds are likely to interfere with the function of trans-acting factors, such as transcription factors, cis-acting elements, such as promoters and enhancers, as well as any class of post-transcriptional, translational or post-translational compounds involved in osteotropic-related disorders. As such, they are powerful candidates for treatment of such disorders, including, but not limited to, localized or disseminated osteosarcoma, lung, colon, melanoma, thyroid, brain, multiple myeloma, and especially including, without limitation, breast and prostate cancers, and benign conditions, such as BPH or arterial sclerotic conditions where calcification occurs. The compounds of the invention additionally can be used to express crucial growth and differentiation associated genes such as growth factors, growth factor receptors, bone morphogenic proteins, etc., for repairing the damages acquired during aging and degenerative conditions.

In one embodiment, the invention provides methods for high throughput screening of compounds that modulate specific expression of genes within osteotropic cells and tissues. In this aspect of the invention, cells from osteotropic-tissues are removed from the transgenic animal and cultured in vitro. The expression of the reporter gene is used to monitor osteotropic-specific gene activity. In a specific embodiment, luciferase is the reporter gene. Compounds identified by this method can be tested further for their effect on osteotropic-related disorders in normal animals.

In another embodiment, the transgenic animal models of the invention can be used for in vivo screening to test the mechanism of action of candidate drugs for their effect on osteotropic-related disorders. Specifically, the effects of the drugs on osteotropic-related disorders including, but not limited to, localized or disseminated osteosarcoma, lung, colon, melanoma, thyroid, brain, multiple myeloma, and especially including, without limitation, breast and prostate cancers, and benign conditions, such as BPH or arterial sclerotic conditions where calcification occurs, can be assayed.

In another embodiment, a gene therapy method for treating and/or preventing osteotropic-related disorders is provided. BSP promoter sequences are used to drive osteotropic-specific expression of toxic or therapeutic molecules and introduced in the osteotropic cells. The method comprises introducing a BSP promoter sequence operatively associated with a nucleic acid encoding a toxic or therapeutic molecule into osteotropic cells. In one embodiment, the invention provides a preventative gene therapy method comprising introducing a BSP promoter sequence operatively associated with a nucleic acid encoding a toxic or therapeutic molecule into osteotropic cells to delay and/or prevent an osteotropic-related disorder. In a specific embodiment, the invention provides a gene therapy method for treatment of cancer or other proliferative disorder, including, but not limited to, localized or disseminated osteosarcoma, lung, colon, melanoma, thyroid, brain, multiple myeloma, and especially including, without limitation, breast and prostate cancers. The BSP promoter sequence is used to direct the expression of one or more proteins specifically in the osteotropic-tumor cells of a patient. In addition, due to the tissue specificity of the promoters used in the present invention, therapeutic and/or toxic agents are effective not only when administered via direct application, such as by injection, but also when administered systemically to the body via intravenous administration, intra arterial administration, intra tumoral administration, perfusion, oral administration or the like, because gene expression will be limited and localized to specific, osteoblastic cell and tissue types. Further, since many of the therapeutic and/or toxic agents of the invention exhibit pleiotropic effects, expression of the therapeutic and/or toxic agents in only specifically targeted cells is essential in order to prevent numerous, harmful side effects.

In addition to tissue specific promoters, the present invention encompasses vectors using inducible promoters. Inducible promoters have the advantage that they can be switched on and off, depending on the clinical state of the patient. Therefore, if a cell is stably transfected with a therapeutic transgene under the control of an inducible promoter, its expression could be controlled over the life-time of an individual.

The invention further provides methods for screening for novel transcription factors that modulate the BSP promoter sequence. Such novel transcription factors identified by this method can be used as targets for treating osteotropic-related disorders.

The invention is also based, in part, on the fact that adenoviral vectors constructed with a BSP transcriptional regulatory sequence described herein are capable of selectively driving expression of an adenovirus gene essential for replication in a tissue specific and tumor-restrictive manner. The invention is further based, in part, on the discovery that such adenoviral vectors can be used as therapeutic agents for treating prostate cancer. Thus, due to the tissue-specificity and tumor-restrictiveness of the BSP transcriptional regulatory sequence used with the adenoviral vectors, the adenovirus can be administered in a tumor-restrictive and tissue-specific manner, with the use of a BSP transcriptional regulatory sequence which allows for tissue specific expression of the adenovirus gene essential for replication and/or heterologous nucleotide sequence. An example of such a BSP transcriptional regulatory sequence is the BSP promoter which is activated only within osteotropic cells and tissues. Thus, an adenovirus vector constructed with an essential gene under the control of an BSP transcriptional regulatory sequence can be expressed effectively and specifically in targeted tumor cells and tissues, thereby minimizing the side effects of expression of the adenovirus vector in non-osteotropic cells and tissues.

In addition, due to the tissue specificity of the BSP transcriptional regulatory sequence used with the adenoviral vectors, the viral vectors of the present invention are effective therapeutic agents not only when administered via direct application, such as by injection, but also when administered systemically to the body via intravenous administration, oral administration or the like, because gene expression will be limited and localized to specific, prostatic cell and disease tissues.

In one embodiment, the invention provides an adenovirus vector comprising an adenovirus with an essential gene under transcriptional control of a BSP transcriptional regulatory sequence. The BSP transcriptional regulatory sequence is capable of mediating gene expression specific to cells which allow a BSP transcriptional regulatory sequence to function, such as for example, tumors and other diseases and disorders involving tumor and tissue cells with calcification potential, including osteotropic-related disorders including, but not limited to, localized or disseminated osteosarcoma, other osteotropic tumors including, but not limited to, lung, colon, melanoma, thyroid, brain, multiple myeloma, and especially including, without limitation, breast and prostate cancers, and benign conditions, such as BPH or arterial sclerotic conditions.

The BSP transcriptional regulatory sequence can comprise a promoter and/or enhancer or enhancer-like sequence from a BSP gene, provided that the BSP transcriptional regulatory sequence is capable of mediating gene expression specific to cells expressing BSP. In one embodiment, a BSP transcriptional regulatory sequence comprises a promoter from a BSP gene. In one embodiment, a BSP transcriptional regulatory sequence comprises an enhancer or enhancer-like sequence from a BSP gene. In one embodiment, a BSP transcriptional regulatory sequence comprises a promoter from a BSP gene and an enhancer or enhancer-like sequence from a BSP gene. In one embodiment, the BSP transcriptional regulatory sequence is transcriptionally active in cells which allow a BSP transcriptional regulatory sequence to function, such as cells expressing BSP.

In some embodiments, the BSP transcriptional regulatory sequence is human, mouse, or rat in origin. In some embodiments, the mouse or rat BSP transcriptional regulatory sequence is capable of mediating prostate-specific gene expression in humans.

In some embodiments, the adenovirus gene under control of a BSP transcriptional regulatory sequence contributes to cytotoxicity (directly or indirectly), such as a gene essential for viral replication. In one embodiment, the adenovirus gene is an early gene. In another embodiment, the early gene is E1A. In another embodiment, the early gene is E1B. In yet another embodiment, both E1A and E1B are under transcriptional control of a BSP transcriptional regulatory sequence. In other embodiments, the adenovirus gene essential for replication is a late gene. In various embodiments, the additional late gene is L1, L2, L3, L4, or L5.

In another embodiment, the adenovirus vector comprising an adenovirus gene under transcriptional control of a BSP transcriptional regulatory sequence further comprises at least one additional adenovirus gene under transcriptional control of at least one additional BSP-specific transcriptional regulatory sequence. In one embodiment, a composition comprises this adenovirus. In one embodiment, this composition further comprises a pharmaceutically acceptable excipient. In one embodiment, the at least one additional BSP-specific transcriptional regulatory sequence is a second BSP transcriptional regulatory sequence. In one embodiment, the at least one additional BSP transcriptional regulatory sequence can have a sequence different from that of the first BSP transcriptional regulatory sequence. In one embodiment, the at least one additional BSP-specific transcriptional regulatory sequence comprises a BSP transcriptional regulatory sequence.

In other embodiments, the adenovirus vector can further comprise a heterologous gene or transgene, wherein said heterologous gene or transgene is under the transcriptional control of a BSP transcriptional regulatory sequence. In one embodiment, the heterologous gene is a reporter gene such as for example, and without limitation, the luciferase reporter gene or beta-galactosidase reporter gene. In one embodiment, the heterologous gene is conditionally required for cell survival. In some embodiments, the transgene is a cytotoxic gene.

In another embodiment, a method of treating metastatic cancer in an individual is provided, the method comprising the step of administering to the individual an effective amount of an adenovirus vector in which an adenovirus gene is under transcriptional control of a BSP transcriptional regulatory sequence, wherein the metastatic cancer is prostate cancer. In another embodiment, a method of treating metastatic cancer in an individual is provided, the method comprising the step of administering to the individual an effective amount of an adenovirus vector in which an adenovirus gene is under transcriptional control of a BSP transcriptional regulatory sequence, wherein the metastatic cancer is prostate cancer.

In one embodiment, the adenovirus gene is essential for viral replication. In one embodiment, the adenovirus gene is an early gene. In one embodiment, the adenovirus gene is E1A. In one embodiment, the adenovirus gene is E1B. In one embodiment, the BSP transcriptional regulatory sequence comprises an enhancer or enhancer-like sequence from a BSP gene. In one embodiment, the BSP transcriptional regulatory sequence comprises a promoter from a BSP gene. In one embodiment, the BSP transcriptional regulatory sequence comprises a promoter from a BSP gene and an enhancer or enhancer-like sequence from a BSP gene. In one embodiment, the adenovirus further comprises an additional adenovirus gene under transcriptional control of at least one additional transcriptional regulatory sequence. In one embodiment, the second transcriptional regulatory sequence comprises a BSP transcriptional regulatory sequence. In one embodiment, the additional adenovirus gene is essential for viral replication. In one embodiment, the additional adenovirus gene is an early gene. In one embodiment, the additional adenovirus gene is E1A. In one embodiment, the additional adenovirus early gene is E1B. In one embodiment, the additional adenovirus gene is a late gene. In various embodiments, the late gene can be L1, L2, L3, L4, or L5.

In another aspect, the invention provides a host cell transformed with any adenovirus vector(s) described herein.

In another aspect, the invention provides a composition comprising an adenovirus vector comprising an adenovirus gene under transcriptional control of a BSP transcriptional regulatory sequence. In one embodiment, the composition further comprises a pharmaceutically acceptable excipient.

In another aspect, the invention provides kits which contain an adenoviral vector(s) described herein.

In another aspect, a method is provided for propagating an adenovirus vector specific for cells which allow a BSP transcriptional regulatory sequence to function, such cells including, for example, prostate cancer cells, prostate stromal cells, breast cancer cells, renal cells, bladder cells, and cells of the endometrium, said method comprising infecting such cells which allow a BSP transcriptional regulatory sequence to function with any of the adenovirus vector(s) described herein, whereby said adenovirus vector is propagated.

In another aspect, a method for modifying the genotype of a target cell is rovided, the method comprising contacting a cell which allows a BSP transcriptional regulatory sequence to function, such cells including, for example, prostate cancer cells, prostate stromal cells, breast cancer cells, renal cells, bladder cells, and cells of the endometrium, with any adenovirus described herein, wherein the adenovirus enters the cell.

In another aspect, methods are provided for detecting cells expressing BSP in a biological sample, comprising contacting cells of a biological sample with an adenovirus vector(s) described herein, and detecting replication of the adenovirus vector, if any.

In one embodiment, a method is provided for detecting cells which allow a BSP transcriptional regulatory sequence to function, for example, prostate cancer cells, prostate stromal cells, breast cancer cells, renal cells, bladder cells, and cells of the endometrium, in a biological sample, the method comprising the steps of: contacting a biological sample with an adenovirus vector comprising an essential adenoviral early or late gene under transcriptional control of a BSP transcriptional regulatory sequence, under conditions suitable for BSP transcriptional regulatory sequence-mediated gene expression in cells which allow a BSP transcriptional regulatory sequence to function; and determining if the BSP transcriptional regulatory sequence mediates gene expression in the biological sample, where BSP transcriptional regulatory sequence-mediated gene expression is indicative of the presence of cells which allow a BSP transcriptional regulatory sequence to function. In one embodiment, the gene is a heterologous (non-adenovirus gene). In one embodiment, the heterologous gene is a reporter gene, and production of the product of the reporter gene is detected.

In another embodiment, a method is provided for conferring selective toxicity or cytotoxicity on a target cell, said method comprising contacting a target cell which allows a BSP transcriptional regulatory sequence to function, for example, without limitation, in prostate cancer cells, prostate stromal cells, breast cancer cells, renal cells, bladder cells, and cells of the endometrium, with any adenovirus disclosed herein, wherein the adenovirus enters the cell.

In yet another embodiment, an adenovirus is provided which comprises a heterologous gene under transcriptional control of a BSP transcriptional regulatory sequence. In one embodiment, the heterologous gene is a reporter gene. In one embodiment, the heterologous gene is conditionally required for cell survival. In one embodiment, a method is provided for detecting cells which allow a BSP transcriptional regulatory sequence to function, such as, for example, without limitation, prostate cancer cells, prostate stromal cells, breast cancer cells, renal cells, bladder cells, and cells of the endometrium, in a sample comprising the steps of: contacting a biological sample with an adenovirus vector comprising a gene under transcriptional control of a BSP transcriptional regulatory sequence, under conditions suitable for BSP transcriptional regulatory sequence-mediated gene expression in cells which allow a BSP transcriptional regulatory sequence to function; and determining if BSP transcriptional regulatory sequence mediates gene expression in the biological sample, where BSP transcriptional regulatory sequence-medicated gene expression is indicative of the presence of cells expressing BSP.

As described in more detail herein, a BSP transcriptional regulatory sequence can comprise any number of configurations, including, but not limited to, a BSP promoter; a BSP enhancer or BSP enhancer-like sequence; a BSP silencer; a BSP promoter and a BSP enhancer or BSP enhancer-like sequence; a BSP promoter and a non-BSP (heterologous) enhancer; a non-BSP (heterologous) promoter and a BSP enhancer or BSP enhancer-like sequence; a non-BSP promoter and multiple copies of enhancers; and multimers of the foregoing. Methods are described herein for measuring the activity of a BSP transcriptional regulatory sequence and thus for determining whether a given cell allows a BSP transcriptional regulatory sequence to function. The promoter and enhancer or BSP enhancer-like sequence of a BSP transcriptional regulatory sequence may be in any orientation and/or distance from the coding sequence of interest, and may comprise multimers of the foregoing, as long as the desired BSP cell-specific transcriptional activity is obtained. Transcriptional activation can be measured in a number of ways known in the art (and as described in more detail below), but is generally measured by detection and/or quantitation of mRNA or the protein product of the coding sequence under control of (i.e., operatively linked to) a BSP transcriptional regulatory sequence. As discussed herein, a BSP transcriptional regulatory sequence can be of varying lengths, and of varying sequence composition.

By “transcriptional activation” or an “increase in transcription”, it is intended that transcription will be increased above basal levels in the target cell (i.e. cells that allow a BSP transcriptional regulatory sequence to function, such as, for example, without limitation, prostate cancer cells, prostate stromal cells, breast cancer cells, renal cells, bladder cells, and cells of the endometrium by at least about 20-fold, more preferably at least about 50-fold, more preferably at least about 100-fold, even more preferably at least about 200-fold, even more preferably at least about 400- to about 500-fold, even more preferably, at least about 1000-fold. Basal levels are generally the level of activity, if any, in a non-BSP-producing cell, or the level of activity (if any) of a reporter construct lacking an BSP transcriptional regulatory sequence as tested in a BSP-producing cell. Optionally, a transcriptional terminator or transcriptional “silencer” can be placed upstream of the BSP transcriptional regulatory sequence, thereby preventing unwanted read-through transcription of the coding segment under transcriptional control of the BSP transcriptional regulatory sequence. Also, optionally, the endogenous promoter of the coding segment to be placed under transcriptional control of the BSP transcriptional regulatory sequence can be deleted.

3.1 Definitions

TK=thymidine kinase;

OC=osteocalcin;

BSP=bone sialoprotein;

AcV=acyclovir;

FBS=fetal bovine serum;

Beta-gal.=beta-galactosidase;

CMV=cytomegalovirus;

ROS 17/2.7=rat osteoblastic osteosarcoma;

MG 63=human osteosarcoma;

NIH 3T3=embryonic mouse fibroblast;

P69=human “normal” prostate cell type without any tumorogenic or metastatic ability;

LNCaP=human androgen dependent prostate cancer;

C4-2=human androgen independent highly tumorogenic/metastasizing prostate cancer;

PC-3M=human androgen independent highly metastatic prostate cancer;

ArCaP=human androgen independent prostate cancer;

Saos-2=human osteosarcoma;

SF/PF=serum free, phenol free

Dl=mouse embryonic pluripotent bone marrow cell;

Lovo=human colon cancer;

MCF-7=human breast cancer;

U-97=human brain cancer of the gioblastoma multiform type;

A547=human lung cancer;

DMEM=Dulbeco's Modified Eagle Media;

T media=prostate cancer cell optimal growth media;

RLU=relative luciferase units;

Dex.=1×10⁻⁷ or 1×10⁻⁸ dexamethasone (glucocorticoid);

Mineral. cond.=50 ug/ml final concentration L-ascorbic acid (vitamin C) and 10 uM final concentration B-glycerol phosphate.

For convenience, the meaning of certain terms and phrases employed in the specification, examples, and appended claims are provided below.

The term “tissue-specific” is intended to mean that the transcriptional regulatory sequence to which the gene essential for viral replication is operably linked functions in that tissue so that replication proceeds in that tissue.

The term “transcriptional regulatory sequence” is used according to its art-recognized meaning. It is intended to mean any DNA sequence which can, by virtue of its sequence, cause the linked gene to be either up- or down-regulated in a particular cell. In one embodiment of the present invention, the native transcriptional regulatory sequence is completely deleted from the vector and replaced with a heterologous transcriptional regulatory sequence. The transcriptional regulatory sequence may be adjacent to the coding region for the gene that is essential for replication, or may be removed from it. Accordingly, in the case of a promoter, the promoter will generally be adjacent to the coding region. In the case of an enhancer or enhancer-like sequence, however, an enhancer or enhancer-like sequence can be found at some distance from the coding region such that there is an intervening DNA sequence between the enhancer or enhancer-like sequence and the coding region. In some cases, the native transcriptional regulatory sequence remains on the vector but is non-functional with respect to transcription of the gene essential for replication. In some cases, the native transcriptional regulatory sequence remains on the vector and is augmented by placement of the tissue-specific tumor-restrictive transcriptional regulatory sequence to which the gene essential for viral replication is operably linked.

An “adenovirus vector” or “adenoviral vector” (used interchangeably) is a term well understood in the art and generally comprises a polynucleotide (defined herein) comprising all or a portion of an adenovirus genome. For purposes of the present invention, an adenovirus vector contains a BSP transcriptional regulatory sequence operably linked to a polynucleotide. The operably linked polynucleotide can be adenoviral or heterologous. An adenoviral vector construct of the present invention can be in any of several forms, including, but not limited to, naked DNA, DNA encapsulated in an adenovirus coat, DNA encapsulated in liposomes, DNA complexed with polylysine, complexed with synthetic polycationic molecules, conjugated with transferrin, and complexed with compounds such as PEG to immunologically “mask” the molecule and/or increase half-life, or conjugated to a non-viral protein. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides. For purposes of this invention, adenovirus vectors are replication-competent in a target cell such as a tumor cell.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P—NH₂) or a mixed phosphoramidate-phosphodiester oligomer. Peyrottes et al. (1996) Nucleic Acids Res. 24:1841-8; Chaturvedi et al. (1996) Nucleic Acids Res. 24:2318-23; Schultz et al. (1996) Nucleic Acids Res. 24:2966-73. A phosphorothiate linkage can be used in place of a phosphodiester linkage. Braun et al. (1988) J. Immunol. 141:2084-9; Latimer et al. (1995) Mol. Immunol. 32:1057-1064. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

A polynucleotide or polynucleotide region has a certain percentage (for example, 80%, 85%, 90%, 95%, 98%, or 99%) of “sequence identity” to another sequence means that, when aligned, that percentage of bases are the same in comparing the two sequences. This alignment and the percent homology or sequence identity can be determined using software programs known in the art, for example, those described in Current Protocols in Molecular Biology (Ausubel et al., eds., 1987), Supp. 30, section 7.7.18, Table 7.7.1. A preferred alignment program is ALIGN Plus (Scientific and Educational Software, Pennsylvania).

As used herein, “a cell which allows a BSP transcriptional regulatory sequence to function”, a cell in which the function of a BSP transcriptional regulatory sequence is “sufficiently preserved”, “a cell in which a BSP transcriptional regulatory sequence functions” is a cell in which a BSP transcriptional regulatory sequence, when operably linked to, for example, a reporter gene, increases expression of the reporter gene at least about 20-fold, more preferably at least about 50-fold, more preferably at least about 100-fold, more preferably at least about 200-fold, even more preferably at least about 400- to 500-fold, even more preferably at least about 1000-fold, when compared to the expression of the same reporter gene when not operably linked to the BSP transcriptional regulatory sequence. Methods for measuring levels (whether relative or absolute) of expression are known in the art and are described herein.

“Under transcriptional control” is a term well-understood in the art and indicates that transcription of a polynucleotide sequence, usually a DNA sequence, depends on its being operably (operatively) linked to an element or transcriptional regulatory sequence which contributes to the initiation of, or promotes, transcription. As noted below, “operably linked” refers to a juxtaposition wherein the elements transcriptional regulatory sequences are in an arrangement allowing them to function.

As used herein, “cytotoxicity” is a term well understood in the art and refers to a state in which one or more of a cell's usual biochemical or biological functions are aberrantly compromised (i.e., inhibited or elevated). These activities include, but are not limited to metabolism; cellular replication; DNA replication; transcription; translation; and uptake of molecules. “Cytotoxicity” includes cell death and/or cytolysis. Assays are known in the art which indicate cytotoxicity, such as dye exclusion, ³H-thymidine uptake, and plaque assays. The term “selective cytotoxicity”, as used herein, refers to the cytotoxicity conferred by an adenovirus vector of the present invention on a cell which allows a BSP transcriptional regulatory sequence to function when compared to the cytotoxicity conferred by the adenovirus on a cell which does not allows a BSP transcriptional regulatory sequence to function. Such cytotoxicity may be measured, for example, by plaque assays, reduction or stabilization in size of a tumor comprising target cells, or the reduction or stabilization of serum levels of a marker characteristic of the tumor cells or a tissue-specific marker, e.g., a cancer marker such as prostate specific antigen.

“Replication” and “propagation” are used interchangeably and refer to the ability of a adenovirus vector of the invention to reproduce or proliferate. This term is well understood in the art. For purposes of this invention, replication involves production of adenovirus proteins and is generally directed to reproduction of adenovirus. Replication can be measured using assays standard in the art and described herein, such as a burst assay or plaque assay. “Replication” and “propagation” include any activity directly or indirectly involved in the process of virus manufacture, including, but not limited to, viral gene expression; production of viral proteins, nucleic acids or other components; packaging of viral components into complete viruses; and cell lysis.

The term “heterologous” means a DNA sequence not found in the native vector genome. With respect to a “heterologous transcriptional regulatory sequence”, “heterologous” indicates that the transcriptional regulatory sequence is not naturally ligated to the DNA sequence for the gene essential for replication of the vector.

A “heterologous gene” or “transgene” is any gene that is not present in wild-type adenovirus. Preferably, the transgene will also not be expressed or present in the target cell prior to introduction by the adenovirus vector. Examples of preferred transgenes are provided below.

The term “promoter” is used according to its art-recognized meaning. It is intended to mean the DNA region, usually upstream to the coding sequence of a gene or operon, which binds RNA polymerase and directs the enzyme to the correct transcriptional start site.

The term “enhancer” is used according to its art-recognized meaning. It is intended to mean a sequence found in eukaryotes and certain eukaryotic viruses which can increase transcription from a gene when located (in either orientation) up to several kilobases from the gene being studied. These sequences usually act as enhancers when on the 5′ side (upstream) of the gene in question. However, some enhancers are active when placed on the 3′ side (downstream) of the gene. The enhancer may also be an enhancer-like sequence.

The term “silencer,” used in its art-recognized sense, means a sequence found in eucaryotic viruses and eucaryotes which can decrease or silence transcription of a gene when located within several kilobases of that gene.

A “heterologous” promoter or enhancer is one which is not associated with or derived from an BSP gene 5′ flanking sequence. Examples of a heterologous promoter are the α-fetoprotein, PSA, DF3, tyrosinase, CEA, surfactant protein, and ErbB2 promoters. Examples of a heterologous enhancer are the α-fetoprotein, PSA, DF3, tyrosinase, CEA, surfactant protein, ErbB2, and SV40 enhancers.

An “endogenous” promoter, enhancer, or transcriptional regulatory sequence is native to or derived from adenovirus.

The term “operably linked” relates to the orientation of polynucleotide elements in a functional relationship. A transcriptional regulatory sequence is operably linked to a coding segment if the transcriptional regulatory sequence promotes transcription of the coding sequence. Operably linked means that the DNA sequences being linked are generally contiguous and, where necessary to join two protein coding regions, contiguous and in the same reading frame. However, since enhancers generally function when separated from the promoter by several kilobases and intronic sequences may be of variable length, some polynucleotide elements may be operably linked but not contiguous.

A “host cell” includes an individual cell or cell culture which can be or has been a recipient of any vector of this invention. Host cells include progeny of a single host cell, and the progeny may not necessarily be completed identical (in morphology or in total DNA complement) to the original parent cell due to natural, accidental, or deliberate mutation and/or change. A host cell includes cells transfected or infected in vivo or in vitro with an adenoviral vector of this invention.

A “target cell” is any cell that allows a BSP transcriptional regulatory sequence to function. Preferably, a target cell is a mammalian cell which allows a BSP transcriptional regulatory sequence to function, such as any cell expressing BSP, preferably, a mammalian cell endogenously expressing BSP, more preferably, a human cell, and more preferably, a human cell capable of allowing a BSP transcriptional regulatory sequence to function.

As used herein, “neoplastic cells”, “neoplasia”, “tumor”, “tumor cells”, “cancer”, and “cancer cells” refer to cells which exhibit relatively autonomous growth, so that they exhibit an aberrant growth phenotype characterized by a significant loss of control of cell proliferation. Neoplastic cells can be benign or malignant.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples.

An “individual” is a vertebrate, preferably a mammal, more preferably a human. Mammals include, but are not limited to, farm animals, sport animals, and pets.

An “effective amount” is an amount sufficient to effect beneficial or desired clinical results. An effective amount can be administered in one or more administrations. For purposes of this invention, an effective amount of an adenoviral vector is an amount that is sufficient to palliate, ameliorate, stabilize, reverse, slow or delay the progression of the disease state.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, preventing spread (i.e., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment. “Palliating” a disease means that the extent and/or undesirable clinical manifestations of a disease state are lessened and/or time course of the progression is slowed or lengthened, as compared to not administering adenoviral vectors of the present invention.

Various combinations of transcriptional regulatory sequences can be included in a vector. One or more may be heterologous. Further, one or more may have the tissue-specificity. On or more of the transcriptional regulatory sequences may be inducible. For example, a single transcriptional regulatory sequence could be used to drive replication by more than one gene essential for replication. This is the case, for example, when the gene product of one of the genes drives transcription of the further gene(s). An example is a heterologous promoter linked to a cassette containing an E1a coding sequence (E1a promoter deleted) and the entire E1b gene. In this instance, only one heterologous transcriptional regulatory sequence may be necessary. When genes are individually (separately) controlled, however, more than one transcriptional regulatory sequence can be used if more than one such gene is desired to control replication.

The term “gene essential for replication” refers to a genetic sequence whose transcription is required for the viral vector to replicate in the target cell.

The vectors of the present invention, therefore, also include transcriptional regulatory sequence combinations wherein there is more than one heterologous transcriptional regulatory sequence, but wherein one or more of these is not tissue-specific or tumor-restrictive. For example, one transcriptional regulatory sequence can be a basal level constitutive transcriptional regulatory sequence. For example, a tissue-specific enhancer or promoter can be combined with a basal level constitutive promoter. In another example, a tissue-specific enhancer or promoter can be combined with an inducible promoter.

### 4 BRIEF DESCRIPTION OF THE FIGURES

The following drawings form part of the present specification and are included to further demonstrate certain aspects of the present invention. The invention may be understood better by reference to one or more of these drawings in combination with the detailed description of specific embodiments presented herein.

FIG. 1: Construction of Ad-BSP-TK, a Recombinant Adenovirus Vector, Containing the Bone Sialoprotein (BSP) Promoter Driven Toxic Thymidine Kinase (TK) Gene. This represents one specific method to utilize the BSP promoter in a gene therapy strategy, although many other delivery vectors (e.g. -liposome, retrovirus, etc.) and therapeutic genes (e.g. -cytosine deaminase, diptheria toxin, cytokines, etc.) can be used to either directly or indirectly(e.g.-by host immune reaction) elicit tumor eradication.

FIG. 2: Determination of Bone Sialoprotein messenger RNA in Various Prostate Cancer Cell Lines.

FIG. 3: Northern Blot Revealing BSP Expression (on the top) in the Prostate Cancer Cells (LNCaP, C4-2, C4-2 B4 and ARCaP), the Human Osteosarcoma Cell Line(MG63), and the Mouse Bone Marrow Cell Line (D1).

FIG. 4: Determination of Reporter Gene (Luciferase) in Target Cells Transiently Infected with the BSP Promoter Driven Recombinant Plasmid.

FIG. 5: BSP Promoter Activity In Vitro under Baseline (T Media, 5% Pbs) Conditions and with an Exogenous Glucocorticoid, Dexamethasone.

FIGS. 6A-6B: BSP Promoter Expression as Rlu in Various Prostate Cancer Cell Lines under Standard Conditions (T Media) and under Mineralization Conditions (With L-ascorbic Acid).

FIG. 7: BSP Promoter-driven Luciferase Activity in Other Cancer Cell Types.

FIG. 8: BSP Promoter Sequence from −2184 to +237.

FIGS. 9A-9C: In Vitro Cytotoxicity Assay with AD-BSP-TK. FIG. 9A represents the assay with the osteosarcoma cell line Saos-2. FIG. 9B represents the assay with the prostate cell line LNCaP. FIG. 9C represents the assay with the prostate cell line C4-2.

FIG. 10: PC-3 Subcutanous Tumor Growth in Nude Mice.

FIG. 11: Construction of a replication-competent type 5 adenovirus, AdBSP-E1, by homologous recombination of a shuttle vector, pBSP-E1, and a recombinant vector, pJM17, in 293 cells.

### 5 DETAILED DESCRIPTION OF THE INVENTION

The present invention provides promoters, enhancers and other regulatory elements that direct expression within osteotropic cells, comprising nucleotide sequences from the 5′ regulatory region, and transcriptionally active fragments thereof, that control expression of a BSP. Specifically provided are expression vectors, host cells and transgenic animals wherein a BSP regulatory region is capable of controlling expression of a heterologous coding sequence, over-expressing an endogenous BSP gene or an inhibitor of a pathological process or knocking out expression of a specific gene believed to be important for a calcification-related disease in tumor and tissue cells with calcification potential.

The invention also provides methods for using said vectors, cells and animals for screening candidate molecules for agonists and antagonists of disorders involving tumor and tissue cells with calcification potential. In an alternate embodiment, the invention provides compositions and methods for modulating expression of compounds within tumor and tissue cells with calcification potential, and to screening compounds that modulate expression within tumor and tissue cells with calcification potential. Methods for using the molecules and compounds identified by the screening assays for therapeutic treatments also are provided.

The invention further provides methods of treating and/or ameliorating tumors and other diseases and disorders with calcification potential, including, but not limited to, localized or disseminated osteosarcoma, lung, colon, melanoma, thyroid, brain, multiple myeloma, breast and prostate cancers. The invention specifically targets sites of metastases of the above mentioned osteotropic tumors, and where applicable, their supporting osseous stroma in the metastatic environment. In addition, the present invention provides therapeutic agents which may be applicable to benign conditions such as benign prostatic hyperplasia (BPH) or arterial sclerotic conditions where calcification occurs.

The present invention additionally relates to methods and compositions for the adenovirus cell therapy. In particular, the compositions of the present invention comprise adenoviral vectors employing a BSP transcriptional regulatory sequence to drive viral replication through the regulation of an adenoviral early gene required for viral replication. The methods of the invention involve use of the adenoviral vectors employing a BSP transcriptional regulatory sequence which drive viral replication through the regulation of an adenoviral early gene required for viral replication to treat metastatic cancers, including, without limitation, tumors and other diseases and disorders with calcification potential, including, but not limited to, localized or disseminated osteosarcoma, lung, colon, melanoma, thyroid, brain, multiple myeloma, breast and prostate cancers.

The invention is based, in part, on the discovery that nucleotide sequences encoding toxic and/or therapeutic coding sequences contained within vectors (i.e. viral vectors) can be administered in a cell and tissue specific manner, with the use of promoters which allow for tissue specific expression of the nucleotide sequences. Further, because the vectors of the invention utilize these promoters to control the expression of toxic and/or therapeutic coding sequences, the vectors of the invention are effective therapeutic agents not only when administered via direct application, but also when administered systemically to the body, because the toxic and/or therapeutic coding sequences will be expressed only in specifically targeted cells, i.e., within tumor and tissue cells with calcification potential.

Taking advantage of this feature, the methods of the present invention are designed to efficiently transfer one or more DNA molecules encoding therapeutic agents to a site where the therapeutic agent is necessary. The methods involve the administration of a vector containing DNA encoding translational products (i.e. therapeutic proteins) or transcriptional products (i.e. antisense or ribozymes) within a mammalian host to a site where the translational product is necessary. Once the vector infects cells where the therapeutic agent is necessary, the coding sequence of interest, i.e., thymidine kinase, is expressed, thereby amplifying the amount of the toxic and/or therapeutic agent, protein or RNA.

The present invention relates also to pharmaceutical compositions comprising vectors containing DNA for use in treating and/or ameliorating osteotropic5 related disorders, including, but not limited to, localized or disseminated osteosarcoma, lung, colon, melanoma, thyroid, brain, multiple myeloma, breast and prostate cancers, and benign conditions, such as, for example, benign prostatic hyperplasia (BPH) or arterial sclerotic conditions where calcification occurs. The compositions of the invention generally are comprised of a bio-compatible material containing the vector containing DNA encoding a therapeutic protein of interest, i.e., thymidine kinase, growth factors, etc. A bio-compatible composition is one that is in a form that does not produce an allergic, adverse or other untoward reaction when administered to a mammalian host.

The invention overcomes shortcomings specifically associated with current recombinant protein therapies for treating and/or ameliorating osteotropic diseases. First, direct gene transfer is a rational strategy that allows transfected cells to (a) make physiological amounts of therapeutic protein, modified in a tissue- and context-specific manner, and (b) deliver this protein to the appropriate cell surface signaling receptor under the appropriate circumstances. Exogenous delivery of such molecules is expected to be associated with significant dosing and delivery problems. Second, repeated administration, while possible, is not required with the methods of the invention because various promoters, including inducible promoters, can be used to control the level of expression of the therapeutic protein of interest. Further, integration of transfected DNA can be associated with long term recombinant protein expression.

The preferred vectors of the present invention are adenoviral vectors. In one preferred embodiment, the adenovirus vector is a human adenovirus. There are a number of different types of adenovirus, such as Ad2, Ad5, and Ad40, which may differ to minor or significant degrees. Particularly, Ad5 and Ad40 differ as to their host cell tropism, as well as the nature of the disease induced by the virus.

In another embodiment, the adenovirus vector for use in the compositions and methods of the invention is canine adenovirus type 1 or canine adenovirus type 2. By way of example, and not by way of limitation, examples of canine adenoviruses that may be used are those described in International Patent Application Numbers WO 91/11525 and WO 94/26914, (the entire contents of each of which are incorporated herein by reference).

In another embodiment, the adenovirus vector for use in the compositions and methods of the invention is a bovine adenovirus. By way of example, and not by way of limitation, an example of a bovine adenovirus is that described in International Patent Application Number WO 95/16048 (the entire contents of which are incorporated herein by reference).

In yet another embodiment, the adenovirus vector for use in the compositions and methods of the invention is ovine adenovirus. By way of example, and not by way of limitation, an example of an ovine adenoviral vector suitable for use in the present invention is the ovine adenovirus OAV287 described in U.S. Pat. No. 6,020,172 (the entire contents of which are incorporated herein by reference).

For the purpose of the subject invention, Ad5 will be exemplified. What follows in Section 5.1 is a brief description of adenovirus-based vectors in general and replication-competent adenovirus vectors in particular.

5.1 ADENOVIRUS-BASED VECTORS

Adenovirus is a large, non-enveloped virus consisting of a dense protein capsid and a large linear (36 kb) double stranded DNA genome. Adenovirus infects a variety of both dividing and non-dividing cells, gaining entry by receptor-mediated uptake into endosomes, followed by internalization. After uncoating, the adenovirus genome expresses a large number of different gene products that are involved in viral replication, modification of host cell metabolism and packaging of progeny viral particles. Three adenovirus gene products are essential for replication of viral genomes: (1) the terminal binding protein which primes DNA replication, (2) the viral DNA polymerase and (3) the DNA binding protein (reviewed in Tamanoi and Stillman, 1983, Immunol. 109:75-87). In addition, processing of the terminal binding protein by the adenovirus 23kDa L3 protease is required to permit subsequent rounds of reinfection (Stillman et al., 1981, Cell, 23:497-508) as well as to process adenovirus structural proteins, permitting completion of self-assembly of capsids (Bhatti and Weber, 1979, Virology, 96:478-485).

Packaging of nascent adenovirus particles takes place in the nucleus, requiring both cis-acting DNA elements and trans-acting viral factors, the latter generally construed to be a number of viral structural polypeptides. Packaging of adenoviral DNA sequences into adenovirus capsids requires the viral genomes to possess functional adenovirus encapsidation signals, which are located in the left and right termini of the linear viral genome (Hearing et al., 1987, J. Virol. 61:2555-2558). Additionally, the packaging sequence must reside near the ends of the viral genome to function (Hearing et al., 1987, J. Virol. 61:2555-2558; Grable and Hearing, 1992, J. Virol., 66:723-731). The E1A enhancer, the viral replication origin and the encapsidation signal compose the duplicated inverted terminal repeat (ITR) sequences located at the two ends of adenovirus genomic DNA. The replication origin is defined loosely by a series of conserved nucleotide sequences in the ITR which must be positioned close to the end of the genome to act as a replication-priming element (reviewed in Challberg and Kelly, 1989, Biochem, 58:671-717; Tamanoi and Stillman, 1983, Immunol. 109:75-87). As shown by several groups, the ITRs are sufficient to confer replication to a heterologous DNA in the presence of complementing adenovirus functions. Adenovirus “mini-chromosomes” consisting of the terminal ITRs flanking short linear DNA fragments (in some cases non-viral DNAs) were found to replicate in vivo at low levels in the presence of infecting wild-type adenovirus, or in vitro at low levels in extracts prepared from infected cells (e.g., Hay et al, 1984, J. Mol. Biol. 175:493-510; Tamanoi and Stillman, 1983, Immunol. 109:75-87).

The expression of foreign genes in “replication-defective” adenoviruses (deleted of region E1) has been exploited for a number of years in many labs, and a variety of published reports describe several different approaches often used in constructing these vectors (Vernon et al, 1991, J. Gen. Virol., 72:1243-1251; Wilkinson and Akrigg, 1992, Nuc. Acids Res., 20:2233-2239; Eloit et al., 1990, J. Gen. Virol., 71:2425-2431; Johnson, 1991; Prevec et al., 1990, J. Infect. Dis., 161:27-30; Haj-Ahmad and Graham, 1986, J. Virol., 57:267-274; Lucito and Schneider, 1992, J. Virol., 66:983-991; reviewed in Graham and Prevec, 1992, Butterworth-Heinemann, 363-393). In general, replication-defective viruses are produced by replacing part, or all, of essential region E1 with a heterologous gene of interest, either by direct ligation to viral genomes in vitro, or by homologous recombination within cells in vivo (procedures reviewed in Berkner, 1992, Curr. Topics Micro. Immunol., 158:39-66). These procedures all produce adenovirus vectors that replicate in complementing cell lines such as 293 cells which provide the El gene products in trans. Replication competent adenovirus vectors also have been described that have the heterologous gene of interest inserted in place of non-essential region E3 (e.g., Haj-Ahmad and Graham, 1986, J. Virol. 57:267-274), or between the right ITR and region E4 (Saito et al., 1985, J. Virol., 54:711-719). In both, replication defective viruses and replication competent viruses, the heterologous gene of interest is incorporated into viral particles by packaging of the recombinant adenovirus genome.

The E1A gene is expressed immediately after viral infection (0-2 hours) and before any other viral genes. E1A protein acts as a trans-acting positive-acting transcriptional regulatory factor, and is required for the expression of the other early viral genes E1B, E2, E3, E4, and the promoter-proximal major late genes. Despite the nomenclature, the promoter proximal genes driven by the major late promoter are expressed during early times after Ad5 infection. Flint (1982) Biochem. Biophys. Acta 651:175-208; Flint (1986) Advances Virus Research 31:169-228; Grand (1987) Biochem J. 241:25-38. In the absence of a functional E1A gene, viral infection does not proceed, because the gene products necessary for viral DNA replication are not produced. Nevins (1989) Adv. Virus Res. 31:35-81. The transcription start site of Ad5 E1A is at n 498 and the ATG start site of the E1A protein is at nt 560 in the virus genome.

The E1B protein functions in trans and is necessary for transport of late mRNA from the nucleus to the cytoplasm. Defects in E1B expression result in poor expression of late viral proteins and an inability to shut off host cell protein synthesis. The promoter of E1B has been implicated as the defining element of difference in the host range of Ad40 and Ad5: clinically Ad40 is an enterovirus, whereas Ad5 causes acute conjunctivitis. Bailey et al. (1993) Virology 193:631; Bailey et al. (1994) Virology 202:695706. E1B proteins are also necessary for the virus to overcome restrictions imposed on viral replication by the host cell cycle and also to reduce the apoptotic effects of E1A. Goodrum et al. (1997) J. Virology 71:548-561. The E1B promoter of Ad5 consists of a single high-affinity recognition site for Sp1 and a TATA box.

The E2 region of adenovirus codes for proteins related to replication of the adenoviral genome, including the 72-kDa DNA-binding protein, the 80-kDa precursor terminal protein and the viral DNA polymerase. The E2 region of Ad5 is transcribed in a rightward orientation from two promoters, termed E2 early and E2 late, mapping at 76.0 and 72.0 map units, respectively. While the E2 late promoter is transiently active during late stages of infection and is independent of the E1A transactivator protein, the E2 early promoter is crucial during the early phases of viral replication.

The E2 early promoter, mapping in Ad5 from 27050-27150, consists of a major and a minor transcription initiation site, the latter accounting for about 5% of the E2 transcripts, two non-canonical TATA boxes, two E2F transcription factor binding sites and an ATF transcription factor binding site. For a detailed review of the E2 promoter architecture see Swaminathan et al., Curr. Topics in Micro. and Immr. (1995) 199 part 3:177-194.

The E2 late promoter overlaps with the coding sequences of a gene encoded by the counterstrand and is therefore not amenable for genetic manipulation. However, the E2 early promoter overlaps only for a few base pairs with sequences coding for a 33 kDa protein on the counterstrand. Notably, the Spel restriction site (Ad5 position 27082) is part of the stop codon for the above mentioned 33 kDa protein and conveniently separates the major E2 early transcription initiation site and TATA-binding protein site from the upstream transcription factor binding sites E2 F and ATF. Therefore, insertion of a BSP transcriptional regulatory sequence having Spel ends into the Spel site in the 1-strand would disrupt the endogenous E2 early promoter of Ad5 and should allow BSP-restricted expression of E2 transcripts.

The E4 gene produces a number of transcription products. The E4 region codes for two polypeptides which are responsible for stimulating the replication of viral genomic DNA and for stimulating late gene expression. The protein products of open reading frames (ORFs) 3 and 6 can both perform these function by binding the 55-kDa protein from E1B and heterodimers of E2F- land DP- 1. The ORF 6 protein requires interaction with the E1B 55-kDa protein for activity while the ORF 3 protein does not. In the absence of functional protein from ORF 3 and ORF 6, plaques are produced with an efficiency less than 10⁻⁶ that of wild type virus. To further restrict viral replication to cells that allow a BSP transcriptional regulatory sequence to function, such as BSP-producing cells, E4 ORFs 1-3 can be deleted, making viral DNA replication and late gene synthesis dependent on E4 ORF 6 protein. By combining such a vector with sequences in which the EIB region is regulated by a BSP transcriptional regulatory sequence, a virus can be obtained in which both the E1B function and E4 function are dependent on a BSP transcriptional regulatory sequence driving E1B.

The major late genes relevant to the subject invention are L1, L2, L3, L4, and L5, which encode proteins of the Ad5 virus virion. All of these genes (typically coding for structural proteins) are probably required for adenoviral replication. The late genes are all under the control of the major late promoter (MLP), which is located in Ad5 at about +5986 to about +6048.

In some embodiments, an BSP transcriptional regulatory sequence is used with an adenovirus gene that is essential for propagation, so that replication-competence is preferentially achievable in the target cell that allow a BSP transcriptional regulatory sequence to function, such as a cell expressing BSP. Preferably, the gene is an early gene, such as E1A, E1B, E2, or E4. (As noted supra, E3 is not essential for viral replication.) More preferably, the early gene under a BSP transcriptional regulatory sequence control is E1A and/or E1B. More than one early gene can be placed under control of a BSP transcriptional regulatory sequence.

In other embodiments, in addition to conferring selective cytotoxic and/or cytolytic activity by virtue of preferential replication competence in cells that allow a BSP transcriptional regulatory sequence to function, such as cells expressing BSP, the adenovirus vectors of this invention can further include a heterologous gene (transgene) under the control of a BSP transcriptional regulatory sequence. In this way, various genetic capabilities may be introduced into target cells allowing a BSP transcriptional regulatory sequence to function, such as cells expressing BSP, particularly cancer cells of prostate cancer. For example, in certain instances, it may be desirable to enhance the degree and/or rate of cytotoxic activity, due to, for example, the relatively refractory nature or particular aggressiveness of the BSP-producing target cell. This could be accomplished by coupling the cell-specific replicative cytotoxic activity with cell-specific expression of, for example, HSV-tk and/or cytosine deaminase (cd), which renders cells capable of metabolizing 55 fluorocytosine (5-FC) to the chemotherapeutic agent 5-fluorouracil (5-FU). Using these types of heterologous genes or transgenes may also confer a bystander effect.

Other desirable transgenes that may be introduced via an adenovirus vector(s) include genes encoding cytotoxic proteins, such as the A chains of diphtheria toxin, ricin or abrin [Palmiter et al. (1987) Cell 50:435; Maxwell et al. (1987) Mol. Cell. Biol. 7:1576; Behringer et al. (1988) Genes Dev. 2:453; Messing et al. (1992) Neuron 8:507; Piatak et al. (1988) J. Biol. Chem. 263:4937; Lamb et al. (1985) Eur. J. Biochem. 148:265; Frankel et al. (1989) Mo. Cell. Biol. 9:415], genes encoding a factor capable of initiating apoptosis, sequences encoding antisense transcripts or ribozymes, which among other capabilities may be directed to mRNAs encoding proteins essential for proliferation, such as structural proteins, or transcription factors; viral or other pathogenic proteins, where the pathogen proliferates intracellularly, genes that encode an engineered cytoplasmic variant of a nuclease (e.g. RNase A) or protease (e.g. awsin, papain, proteinase K, carboxypeptidase, etc.), or encode the Fas gene, and the like. Other genes of interest include cytokins, antigens, transmembrane proteins, and the like, such as IL-1, -2, -6, -12, GM-CSF, G-CSF, M-CSF, IFN-.alpha., -.beta., -.gamma., TNF-.alpha., -.beta., NGF, and the like. The positive effector genes could be used in an early phase, followed by cytotoxic activity due to replication.

In alternative embodiments, adenovirus vectors are provided with any of the other genes essential for replication, such as, for example, but not limited to, E2 or E4, under the control of a heterologous transcriptional regulatory sequence.

With respect to the packaging capacity of the adenovirus vectors of the present invention, if no adenovirus sequences have been deleted, an adenoviral vector can be packaged with extra sequences totaling up to about 5% of the genome size, or approximately 1.8 kb. If non-essential sequences are removed from the adenovirus genome, then an additional 4.6 kb of insert can be accommodated (i.e., a total of about 1.8 kb plus 4.6 kb, which is about 6.4 kb). Examples of non-essential adenoviral sequences that can be deleted are E3 and E4 (as long as the E4 ORF6 is maintained).

Described in detail below, in Sections 5.2 and 5.3, are nucleotide sequences of the BSP regulatory region, and expression vectors, host cells and transgenic animals wherein the expression of a heterologous coding sequence is controlled by the BSP regulatory region. In Section 5.4, methods for using such polynucleotides (i.e., regulatory regions of the BSP gene) and fusion protein products, for screening compounds that interact with the regulatory region of the BSP gene are described. This Section describes both in vivo and in vitro assays to screen small molecules, compounds, recombinant proteins, peptides, nucleic acids, antibodies, etc. which bind to or modulate the activity of the BSP regulatory region. Section 5.5 describes methods for the use of the compositions of the invention, identified agonists and antagonists for drug delivery or gene therapy. Finally, in Section 5.6, pharnaceutical compositions are described for using such compositions, agonists and antagonists to modulate osteotropic-related disorders. Methods and compositions are provided for treating various osteotropic-related disorders, including, but not limited to, localized or disseminated osteosarcoma, lung, colon, melanoma, thyroid, brain, multiple myeloma, breast and prostate cancers, and benign conditions, including, but not limited to, benign prostatic hyperplasia (BPH) or arterial sclerotic conditions where calcification occurs.

5.2 Polynucleotides and Nucleic Acids of the Invention

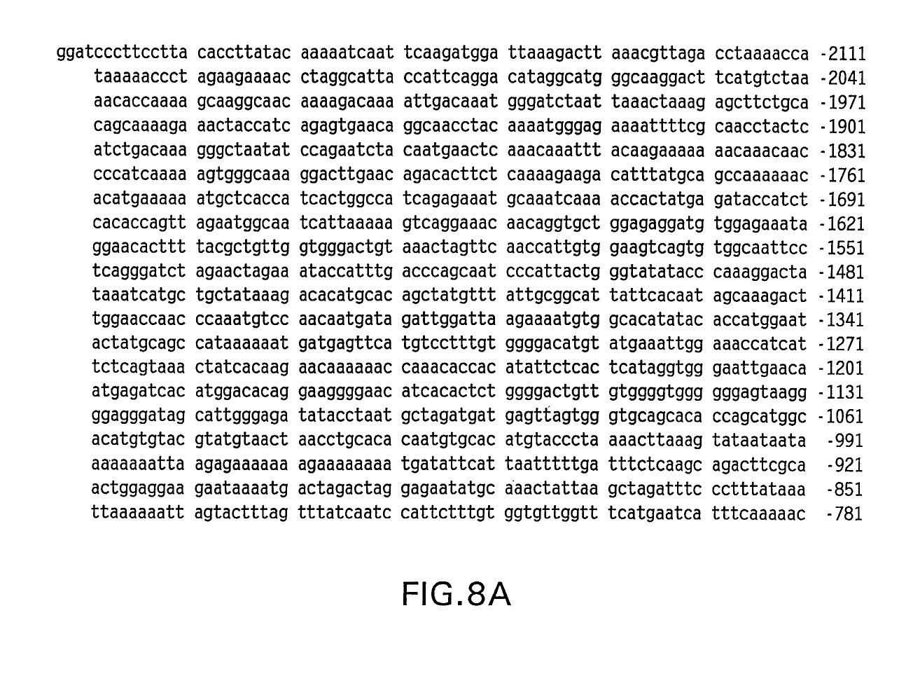

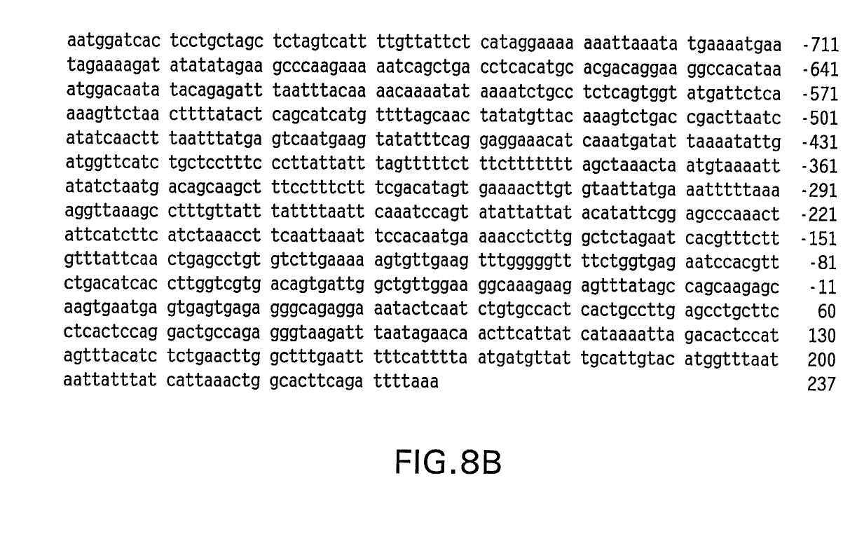

The present invention encompasses polynucleotide sequences comprising the 5′ regulatory region, and transcriptionally active fragments thereof, of the BSP gene. In particular, the present invention provides polynucleotides comprising 907 bp, 1107 bp, 1418 bp, 1459 bp and 2253 bp sequences that are located within a BSP gene. Specifically, the polynucleotides comprise −838 bp through +69 bp, −1038 bp through +69 bp, −1349 bp through +69 bp, −1390 bp through +69 bp and −2184 bp through +69 bp, respectively, of the BSP sequence shown in FIG. 8. In various embodiments, the polynucleotide may be 5000, 4000, 3000, 2000, 1000 and preferably approximately 500 bp in length.

The invention further provides probes, primers and fragments of the BSP regulatory region. In one embodiment, purified nucleic acids consisting of at least 8 nucleotides (i.e., a hybridizable portion) of a BSP gene sequence are provided; in other embodiments, the nucleic acids consist of at least 20 (contiguous) nucleotides, 25 nucleotides, 50 nucleotides, 100 nucleotides, 200 nucleotides, 500, 1000, 2000, 3000, 4000 or 5000 nucleotides of a BSP sequence. Methods which are well known to those skilled in the art can be used to construct these sequences, either in isolated form or contained in expression vectors. These methods include, for example, in vitro recombinant DNA techniques, synthetic techniques and in vivo genetic recombination. See, e.g., the techniques described in Sambrook et al., 1989, supra, and Ausabel et al., 1989, supra; also see the techniques described in “Oligonucleotide Synthesis”, 1984, Gait M. J. ed., IRL Press, Oxford, which is incorporated herein by reference in its entirety.

In another embodiment, the nucleic acids are smaller than 20, 25, 35, 200 or 500 nucleotides in length. Nucleic acids can be single or double stranded. The invention also encompasses nucleic acids hybridizable to or complementary to the foregoing sequences. In specific aspects, nucleic acids are provided which comprise a sequence complementary to at least 10, 20, 25, 50, 100, 200, 500 nucleotides or the entire regulatory region of a BSP gene.

The probes, primers and fragments of the BSP regulatory region provided by the present invention can be used by the research community for various purposes. They can be used as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; and as a probe to “subtract-out” known sequences in the process of discovering other novel polynucleotides. Methods for performing the uses listed above are well known to those skilled in the art. References disclosing such methods include, without limitation, “Molecular Cloning: A Laboratory Manual”, 2nd ed., Cold Spring Harbor Laboratory Press, Sambrook, J., E. F. Fritsch and T.

Maniatis eds., 1989, and “Methods in Enzymology: Guide to Molecular Cloning Techniques”, Academic Press, Berger, S. L. and A. R. Kimmel eds., 1987.

The nucleotide sequences of the invention also include nucleotide sequences that have at least 65%, 70%, 75%, 80%, 85%, 90%, 95%, 98%, 99% or more nucleotide sequence identity to the nucleotide sequence depicted in FIG. 8, and/or transcriptionally active fragments thereof, which are capable of driving expression specifically within tumor and tissue cells with calcification potential.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity =# of identical overlapping positions/total # of positions ×100). In one embodiment, the two sequences are the same length.

The determination of percent identity between two sequences also can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score =100, wordlength =12 to obtain nucleotide sequences homologous to a nucleic acid molecules of the invention. BLAST protein searches can be performed with the XBLAST program, score =50, wordlength =3 to obtain amino acid sequences homologous to a protein molecules of the invention. To obtain gapped alignments for comparison purposes, Gapped BLAST can be utilized as described in Altschul et al. (1997) Nucleic Acids Res.25:3389-3402. Alternatively, PSI-Blast can be used to perform an iterated search which detects distant relationships between molecules (Id.). When utilizing BLAST, Gapped BLAST and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12 and a gap penalty of 4 can be used. In an alternate embodiment, alignments can be obtained using the NA_MULTIPLE_ALIGNMENT 1.0 program, using a GapWeight of 5 and a GapLengthWeight of 1.

The percent identity between two sequences can be determined using techniques similar to those described above, with or without allowing gaps. In calculating percent identity, typically only exact matches are counted.

The invention also encompasses:

(a) DNA vectors that contain any of the foregoing BSP regulatory sequences and/or their complements (i.e., antisense);

(b) DNA expression vectors that contain any of the foregoing BSP regulatory element sequences operatively associated with a heterologous gene, such as a reporter gene; and

(c) genetically engineered host cells that contain any of the foregoing BSP regulatory element sequences operatively associated with a heterologous gene such that the BSP regulatory element directs the expression of the heterologous gene in the host cell.

Also encompassed within the scope of the invention are various transcriptionally active fragments of this regulatory region. A “transcriptionally active” or “transcriptionally functional” fragment of the sequence depicted in FIG. 8 according to the present invention refers to a polynucleotide comprising a fragment of said polynucleotide which is functional as a regulatory region for expressing a recombinant polypeptide or a recombinant polynucleotide in a recombinant cell host. For the purpose of the invention, a nucleic acid or polynucleotide is “transcriptionally active” as a regulatory region for expressing a recombinant polypeptide or a recombinant polynucleotide if said regulatory polynucleotide contains nucleotide sequences which contain transcriptional information, and such sequences are operably associated to nucleotide sequences which encode the desired polypeptide or the desired polynucleotide.

In particular, the transcriptionally active fragments of the BSP regulatory region of the present invention encompass those fragments that are of sufficient length to promote transcription of a heterologous gene, such as a reporter gene, when operatively linked to the BSP regulatory sequence and transfected into tumor and tissue cells with calcification potential. Typically, the regulatory region is placed immediately 5′ to, and is operatively associated with the coding sequence. As used herein, the term “operatively associated” refers to the placement of the regulatory sequence immediately 5′ (upstream) of the reporter gene, such that transacting factors required for initiation of transcription, such as transcription factors, polymerase subunits and accessory proteins, can assemble at this region to allow RNA polymerase dependent transcription initiation of the reporter gene.

In one embodiment, the polynucleotide sequence chosen may further comprise other nucleotide sequences, either from the BSP gene, or from a heterologous gene. In another embodiment, multiple copies of a promoter sequence, or a fragment thereof, may be linked to each other. For example, the promoter sequence, or a fragment thereof, may be linked to another copy of the promoter sequence, or another fragment thereof, in a head to tail, head to head, or tail to tail orientation. In another embodiment, an osteotropic-specific enhancer may be operatively linked to the BSP regulatory sequence, or fragment thereof, and used to enhance transcription from the construct containing the BSP regulatory sequence.

Also encompassed within the scope of the invention are modifications of this nucleotide sequence without substantially affecting its transcriptional activities. Such modifications include additions, deletions and substitutions. In addition, any nucleotide sequence that selectively hybridizes to the complement of the sequence depicted in FIG. 8 under stringent conditions, and is capable of activating the expression of a coding sequence specifically within tumor and tissue cells with calcification potential is encompassed by the invention. Exemplary moderately stringent hybridization conditions are as follows: prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.01% PVP, 0.01% Ficoll, and 0.01% BSA. This is followed by a wash in 0.1×SSC at 50° C. for 45 min before autoradiography. Alternatively, exemplary conditions of high stringency are as follows: e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & sons, Inc., New York, at p. 2.10.3). Other conditions of high stringency which may be used are well known in the art. In general, for probes between 14 and 70 nucleotides in length the melting temperature (TM) is calculated using the formula: Tm(° C.)=81.5+16.6(log[monovalent cations (molar)])+0.41(% G+C)−(500/N) where N is the length of the probe. If the hybridization is carried out in a solution containing formamide, the melting temperature is calculated using the equation Tm(° C.)=81.5+16.6(log[monovalent cations (molar)])+0.41(% G+C)−(0.6 1% formamide)(500/N) where N is the length of the probe. In general, hybridization is carried out at about 20-25 degrees below Tm (for DNA-DNA hybrids) or 10-15 degrees below Tm (for RNADNA hybrids).

The BSP regulatory region, or transcriptionally functional fragments thereof, is preferably derived from a mammalian organism. Screening procedures which rely on nucleic acid hybridization make it possible to isolate gene sequences from various organisms. The isolated polynucleotide sequence disclosed herein, or fragments thereof, may be labeled and used to screen a cDNA library constructed from mRNA obtained from appropriate cells or tissues (e.g., calcified tissue) derived from the organism of interest. The hybridization conditions used should be of a lower stringency when the cDNA library is derived from an organism different from the type of organism from which the labeled sequence was derived. Low stringency conditions are well known to those of skill in the art, and will vary depending on the specific organisms from which the library and the labeled sequence are derived. For guidance regarding such conditions see, for example, Sambrook et al., 1989, Molecular Cloning, A Laboratory Manual, Second Edition, Cold Spring Harbor Press, N.Y., and Ausabel et al., 1989, Current Protocols in Molecular Biology, Green Publishing Associates and Wiley Interscience, N.Y., each of which is incorporated herein by reference in its entirety. Further, mammalian BSP regulatory region homologues may be isolated from, for example, bovine or other non-human nucleic acid, by performing polymerase chain reaction (PCR) amplification using two primer pools designed on the basis of the nucleotide sequence of the BSP regulatory region disclosed herein. The template for the reaction may be cDNA obtained by reverse transcription of the mRNA prepared from, for example, bovine or other non-human cell lines, or tissue known to express the BSP gene. For guidance regarding such conditions, see, e.g., Innis et al. (Eds.) 1995, PCR Strategies, Academic Press Inc., San Diego; and Erlich (ed) 1992, PCR Technology, Oxford University Press, New York, each of which is incorporated herein by reference in its entirety.

Promoter sequences within the 5′ non-coding regions of the BSP gene may be further defined by constructing nested 5′ and/or 3′ deletions using conventional techniques such as exonuclease III or appropriate restriction endonuclease digestion. The resulting deletion fragments can be inserted into the promoter reporter vector to determine whether the deletion has reduced or obliterated promoter activity, such as described, for example, by Coles et al. (Hum. Mol. Genet., 7:791-800, 1998). In this way, the boundaries of the promoters may be defined. If desired, potential individual regulatory sites within the promoter may be identified using site directed mutagenesis or linker scanning to obliterate potential transcription factor binding sites within the promoter individually or in combination. The effects of these mutations on transcription levels may be determined by inserting the mutations into cloning sites in promoter reporter vectors. These types of assays are well known to those skilled in the art (WO 97/17359, U.S. Pat. No. 5,374,544, EP 582 796, U.S. Pat. No. 5,698,389, U.S. Pat. No. 5,643,746, U.S. Pat. No. 5,502,176, and U.S. Pat. No. 5,266,488).

The BSP regulatory regions and transcriptionally functional fragments thereof, and the fragments and probes described herein which serve to identify BSP regulatory regions and fragments thereof, may be produced by recombinant DNA technology using techniques well known in the art. Methods which are well known to those skilled in the art can be used to construct these sequences, either in isolated form or contained in expression vectors. These methods include, for example, in vitro recombinant DNA techniques, synthetic techniques and in vivo genetic recombination. See, e.g., the techniques described in Sambrook et al., 1989, supra, and Ausabel et al, 1989, supra; also see the techniques described in “Oligonucleotide Synthesis”, 1984, Gait M. J. ed., IRL Press, Oxford, which is incorporated herein by reference in its entirety.

Alterations in the regulatory sequences can be generated using a variety of chemical and enzymatic methods which are well known to those skilled in the art. For example, regions of the sequences defined by restriction sites can be deleted. Oligonucleotide-directed mutagenesis can be employed to alter the sequence in a defined way and/or to introduce restriction sites in specific regions within the sequence. Additionally, deletion mutants can be generated using DNA nucleases such as Bal31, ExoIII, or S1 nuclease. Progressively larger deletions in the regulatory sequences are generated by incubating the DNA with nucleases for increased periods of time (see, e.g., Ausubel et al., 1989, supra).

The altered sequences are evaluated for their ability to direct expression of heterologous coding sequences in appropriate host cells. It is within the scope of the present invention that any altered regulatory sequences which retain their ability to direct expression of a coding sequence be incorporated into recombinant expression vectors for further use.

5.3 Analysis of Osteotropic-specific Promoter Activity

The BSP regulatory region shows selective tissue and cell-type specificity; i.e., it induces gene expression in osteotropic cells. Thus, the regulatory region, and transcriptionally active fragments thereof, of the present invention may be used to induce expression of a heterologous coding sequence specifically in osteotropic cells. The present invention provides for the use of the BSP regulatory region to achieve tissue specific expression of a target coding sequence. The activity and the specificity of the BSP regulatory region can further be assessed by monitoring the expression level of a detectable polynucleotide operably associated with the BSP promoter in different types of cells, tissues and cell lines engineered to contain the BSP promoter. As discussed hereinbelow, the detectable polynucleotide may be either a polynucleotide that specifically hybridizes with a predefined oligonucleotide probe, or a polynucleotide encoding a detectable protein.

5.3.1 BSP Promoter Driven Reporter Constructs

The regulatory polynucleotides according to the invention may be advantageously part of a recombinant expression vector that may be used to express a coding sequence, or reporter gene, in a desired host cell or host organism. The BSP regulatory region of the present invention, and transcriptionally active fragments thereof, may be used to direct the expression of a heterologous coding sequence. In particular, the present invention encompasses mammalian BSP regulatory regions. In accordance with the present invention, transcriptionally active fragments of the BSP regulatory region encompass those fragments of the region which are of sufficient length to promote transcription of a reporter coding sequence to which the fragment is operatively linked.

A variety of reporter gene sequences well known to those of skill in the art can be utilized, including, but not limited to, genes encoding fluorescent proteins such as green fluorescent protein (GFP), enzymes (e.g. CAT, beta-galactosidase, luciferase) or antigenic markers. For convenience, enzymatic reporters and light-emitting reporters analyzed by colorometric or fluorometric assays are preferred for the screening assays of the invention.

In one embodiment, for example, a bioluminescent, chemiluminescent or fluorescent protein can be used as a light-emitting reporter in the invention. Types of light-emitting reporters, which do not require substrates or cofactors, include, but are not limited to the wild-type green fluorescent protein (GFP) of Victoria aequoria (Chalfie et al., 1994, Science 263:802-805), and modified GFPs (Heim et al., 1995, Nature 373:663-4; PCT publication WO 96/23810). Transcription and translation of this type of reporter gene leads to the accumulation of the fluorescent protein in test cells, which can be measured by a fluorimeter, or a flow cytometer, for example, by methods that are well known in the art (see, e.g., Lackowicz, 1983, Principles of Fluorescence Spectroscopy, Plenum Press, New York).

Another type of reporter gene that may be used are enzymes that require cofactor(s) to emit light, including but not limited to, Renilla luciferase. Other sources of luciferase also are well known in the art, including, but not limited to, the bacterial luciferase (luxAB gene product) of Vibrio harveyi (Karp, 1989, Biochim. Biophys. Acta 1007:84-90; Stewart et al. 1992, J. Gen. Microbiol, 138:1289-1300), and the luciferase from firefly, Photinus pyralis (De Wet et al. 1987, Mol. Cell. Biol. 7:725-737), which can be assayed by light production (Miyamoto et al., 1987, J. Bacteriol. 169:247-253; Loessner et al 1996, Environ. Microbiol. 62:1133-1140; and Schultz & Yarus, 1990, J. Bacteriol. 172:595-602).

Reporter genes that can be analyzed using colorimetric analysis include, but are not limited to, β-galactosidase (Nolan et al. 1988, Proc. Natl. Acad. Sci. USA 85:260307), β-glucuronidase (Roberts et al. 1989, Curr. Genet. 15:177-180), luciferase (Miyamoto et al., 1987, J. Bacteriol. 169:247-253), or β-lactamase. In one embodiment, the reporter gene sequence comprises a nucleotide sequence which encodes a LacZ gene product, Pgalactosidase. The enzyme is very stable and has a broad specificity so as to allow the use of different histochemical, chromogenic or fluorogenic substrates, such as, but not limited to, 5-bromo-4-chloro-3-indoyl-β-D-galactoside (X-gal), lactose 2,3,5-triphenyl-2H-tetrazolium (lactose-tetrazolium) and fluorescein galactopyranoside (see Nolan et al., 1988, supra).

In another embodiment, the product of the E. coli β-glucuronidase gene (GUS) can be used as a reporter gene (Roberts et al. 1989, Curr. Genet. 15:177-180). GUS activity can be detected by various histochemical and fluorogenic substrates, such as Xglucuronide (Xgluc) and 4-methylumbelliferyl glucuronide.

In addition to reporter gene sequences such as those described above, which provide convenient colorimetric responses, other reporter gene sequences, such as, for example, selectable reporter gene sequences, can routinely be employed. For example, the coding sequence for chloramphenicol acetyl transferase (CAT) can be utilized, leading to BSP regulatory region-dependent expression of chloramphenicol resistant cell growth. The use of CAT and the advantages of a selectable reporter gene are well known to those skilled in the art (Eikmanns et al. 1991, Gene 102:93-98). Other selectable reporter gene sequences also can be utilized and include, but are not limited to, gene sequences encoding polypeptides which confer zeocin (Hegedus et al. 1998, Gene 207:241-249) or kanamycin resistance (Friedrich & Soriano, 1991, Genes. Dev. 5:1513-1523).

Other coding sequences, such as toxic gene products, potentially toxic gene products, and antiproliferation or cytostatic gene products, also can be used. In another embodiment, the detectable reporter polynucleotide may be either a polynucleotide that specifically hybridizes with a predefined oligonucleotide probe, or a polynucleotide encoding a detectable protein, including a BSP polypeptide or a fragment or a variant thereof. This type of assay is well known to those skilled in the art (U.S. Pat. No. 5,502,176 and U.S. Pat. No. 5,266,488).

BSP promoter driven reporter constructs can be constructed according to standard recombinant DNA techniques (see, e.g., Methods in Enzymology, 1987, volume 154, Academic Press; Sambrook et al. 1989, Molecular Cloning—A Laboratory Manual, 2nd Edition, Cold Spring Harbor Press, New York; and Ausubel et al. Current Protocols in Molecular Biology, Greene Publishing Associates and Wiley Interscience, New York, each of which is incorporated herein by reference in its entirety).

Methods for assaying promoter activity are well-known to those skilled in the art (see, e.g., Sambrook et al., Molecular Cloning A Laboratory Manual, Cold Spring Harbor Laboratory, Cold Spring Harbor, N.Y., 1989). An example of a typical method that can be used involves a recombinant vector carrying a reporter gene and genomic sequences from the BSP sequence depicted in FIG. 8. Briefly, the expression of the reporter gene (for example, green fluorescent protein, luciferase, β-galactosidase or chloramphenicol acetyl transferase) is detected when placed under the control of a biologically active polynucleotide fragment. Genomic sequences located upstream of the first exon of the gene may be cloned into any suitable promoter reporter vector. For example, a number of commercially available vectors can be engineered to insert the BSP regulatory region of the invention for expression in mammalian host cells. Non-limiting examples of such vectors are pSEAPBasic, pSEAP-Enhancer, ppgal-Basic, pβgal-Enhancer, or pEGFP-1 Promoter Reporter vectors (Clontech, Palo Alto, Calif.) or pGL2-basic or pGL3-basic promoterless luciferase reporter gene vector (Promega, Madison, Wis.). Each of these promoter reporter vectors include multiple cloning sites positioned upstream of a reporter gene encoding a readily assayable protein such as secreted alkaline phosphatase, green fluorescent protein, luciferase or β-galactosidase. The regulatory sequences of the BSP gene are inserted into the cloning sites upstream of the reporter gene in both orientations and introduced into an appropriate host cell. The level of reporter protein is assayed and compared to the level obtained with a vector lacking an insert in the cloning site. The presence of an elevated expression level in the vector containing the insert with respect the control vector indicates the presence of a promoter in the insert.

Expression vectors that comprise a BSP regulatory region may further contain a gene encoding a selectable marker. A number of selection systems may be used, including but not limited to, the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026) and adenine phosphoribosyltransferase (Lowy et al., 1980, Cell 22:817) genes, which can be employed in tk⁻, hgprt⁻ or aprt⁻ cells, respectively. Also, antimetabolite resistance can be used as the basis of selection for dhfr, which confers resistance to methotrexate (Wigler et al., 1980, Proc. Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes. Additional selectable genes include trpB, which allows cells to utilize indole in place of tryptophan; hisD, which allows cells to utilize histinol in place of histidine (Hartman & Mulligan, 1988, Proc. Natl. Acad. Sci. USA 85:8047); ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.) and glutamine synthetase (Bebbington et al., 1992, Biotech 10:169).

5.3.2 Characterization of Transcriptionally Active Regulatory Fragments

A fusion construct comprising a BSP regulatory region, or a fragment thereof, can be assayed for transcriptional activity. As a first step in promoter analysis, the transcriptional start point (+1 site) of the osteotropic-specific gene under study has to be determined using primer extension assay and/or RNAase protection assay, following standard methods (Sambrook et al.,1989, Molecular Cloning: A Laboratory Manual, Cold Spring Harbor, Cold Spring Harbor Press). The DNA sequence upstream of the +1 site is generally considered as the promoter region responsible for gene regulation. However, downstream sequences, including sequences within introns, also may be involved in gene regulation. To begin testing for promoter activity, a −3 kb to +3 kb region (where +1 is the transcriptional start point) may be cloned upstream of the reporter gene coding region. Two or more additional reporter gene constructs also may be made which contain 5′ and/or 3′ truncated versions of the regulatory region to aid in identification of the region responsible for osteotropic-specific expression. The choice of the type of reporter gene is made based on the application.

In a preferred embodiment, a GFP reporter gene construct is used. The application of green fluorescent protein (GFP) as a reporter is particularly useful in the study of osteotropic-specific gene promoters. A major advantage of using GFP as a reporter lies in the fact that GFP can be detected in freshly isolated tumor and tissue cells with calcification potential without the need for substrates.

In another embodiment of the invention, a Lac Z reporter construct is used. The Lac Z gene product, β-galactosidase, is extremely stable and has a broad specificity so as to allow the use of different histochemical, chromogenic or fluorogenic substrates, such as, but not limited to, 5-bromo-4-chloro-3-indoyl-β-D-galactoside (X-gal), lactose 2,3,5-triphenyl-2H-tetrazolium (lactose-tetrazolium) and fluorescein galactopyranoside (see Nolan et al., 1988, supra).

For promoter analysis in transgenic mice, GFP that has been optimized for expression in mammalian cells is preferred. The promoterless cloning vector pEGFP1(Clontech, Palo Alto, Calif.) encodes a red shifted variant of the wild-type GFP which has been optimized for brighter fluorescence and higher expression in mammalian cells (Cormack et al., 1996, Gene 173:33; Haas et al., 1996, Curr. Biol. 6:315). Moreover, since the maximal excitation peak of this enhanced GFP (EGFP) is at 488 nm, commonly used filter sets such as fluorescein isothiocyanate (FITC) optics which illuminate at 450-500 nm can be used to visualize GFP fluorescence. pEGFP1 proved to be useful as a reporter vector for promoter analysis in transgenic mice (Okabe et al, 1997, FEBS Lett. 407:313). In an alternate embodiment, transgenic mice containing transgenes with a BSP regulatory region upstream of a luciferase reporter gene are utilized.

Putative promoter fragments can be prepared (usually from a parent phage clone containing 8-10 kb genomic DNA including the promoter region) for cloning using methods known in the art. In one embodiment, for example, promoter fragments are cloned into the multiple cloning site of a luciferase reporter vector. In one embodiment, restriction endonucleases are used to excise the regulatory region fragments to be inserted into the reporter vector. However, the feasibility of this method depends on the availability of proper restriction endonuclease sites in the regulatory fragment. In a preferred embodiment, the required promoter fragment is amplified by polymerase chain reaction (PCR; Saiki et al., 1988, Science 239:487) using oligonucleotide primers bearing the appropriate sites for restriction endonuclease cleavage. The sequence necessary for restriction cleavage is included at the 5′ end of the forward and reverse primers which flank the regulatory fragment to be amplified. After PCR amplification, the appropriate ends are generated by restriction digestion of the PCR product. The promoter fragments, generated by either method, are then ligated into the multiple cloning site of the reporter vector following standard cloning procedures (Sambrook et al.,1989, supra). It is recommended that the DNA sequence of the PCR generated promoter fragments in the constructs be verified prior to generation of transgenic animals. The resulting reporter gene construct will contain the putative promoter fragment located upstream of the reporter gene open reading frame, e.g., GFP or luciferase cDNA.

In a preferred embodiment, the following protocol is used. Fifty to 100 pg of the reporter gene construct is digested using appropriate restriction endonucleases to release the transgene fragment. The restriction endonuclease cleaved products are resolved in a 1% (w/v) agarose gel containing 0.5 ug/ml ethidium bromide and TAE buffer (IX: 0.04 M Triacetate, 0.001 M EDTA, pH 8.0) at 5-6 V/cm. The transgene band is located by size using a UV transilluminator, preferably using long-wavelength UV lamp to reduce nicking of DNA, and the gel piece containing the required band carefully excised. The gel slice and 1 ml of 0.5×TAE buffer is added to a dialysis bag, which has been boiled in 1 mM EDTA, pH 8.0 for 10 minutes (Sambrook et al.,1989, supra) and the ends are fastened. The dialysis bag containing the gel piece is submerged in a horizontal gel electrophoresis chamber containing 0.5×TAE buffer, and electrophoresed at 5-6 V/cm for 45 minutes. The current flow in the electrophoresis chamber is reversed for one minute before stopping the run to release the DNA which may be attached to the wall of the dialysis tube. The TAE buffer containing the electroeluted DNA from the dialysis bag is collected in a fresh eppendorf tube. The gel piece may be observed on the UV transilluminator to ascertain that the electroelution of the DNA is complete.

The electroeluted DNA sample is further purified by passing through Elutip D columns. The matrix of the column is prewashed with 1-2 ml of High salt buffer (1.0 M NaCl, 20 mM Tris. Cl, 1.0 mM EDTA, pH 7.5), followed by a wash with 5 ml of Low salt buffer (0.2 M NaCl, 20 mM Tris. Cl, 1.0 mM EDTA, pH 7.5). A 5 ml syringe is used to apply solutions to the Elutip D column, avoiding reverse flow. The solution containing the electroeluted DNA is loaded slowly. The column is washed with 2-3 ml of Low salt buffer and the DNA is eluted in 0.4 ml of High salt buffer. Two volumes of cold 95% ethanol is added to precipitate DNA. The DNA is collected by centrifugation in a microcentrifuge at 14,000 g for 10 minutes, carefully removing the alcohol without disrupting the DNA pellet. The pellet is washed at least twice with 70% (v/v) ethanol, and dried. The washing and drying steps are important, as residual salt and ethanol are lethal to the developing embryos. The DNA is resuspend in the injection buffer (10 mM TM, 0.1 mM EDTA, pH 7.5 prepared with Milli-Q quality water). The concentration of the purified transgene DNA fragment is determined by measuring the optical density at A₂₆₀ (A₂₆₀=1 for 50 μg/ml DNA) using a spectrophotometer. DNA prepared in this manner is suitable for microinjection into fertilized mouse eggs.

5.3.3 Osteotropic-specific Promoter Analysis Using Transgenic Mice

The mammalian BSP regulatory region can be used to direct expression of, inter alia, a reporter coding sequence, a homologous gene or a heterologous gene in transgenic animals specifically within tumor and tissue cells with calcification potential. Animals of any species, including, but not limited to, mice, rats, rabbits, guinea pigs, pigs, micro-pigs, goats, sheep, and non-human primates, e.g., baboons, monkeys and chimpanzees may be used to generate transgenic animals. The term “transgenic,” as used herein, refers to non-human animals expressing BSP gene sequences from a different species (e.g., mice expressing human BSP sequences), as well as animals that have been genetically engineered to over-express endogenous (i.e., same species) BSP sequences or animals that have been genetically engineered to knock-out specific sequences.

In one embodiment, the present invention provides for transgenic animals that carry a transgene such as a reporter gene, therapeutic and/or toxic coding sequence under the control of the BSP regulatory region, or transcriptionally active fragments thereof, in all their cells, as well as animals that carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (1992, Proc. Natl. Acad. Sci. USA 89:6232-6236). When it is desired that the transgene be integrated into the chromosomal site of the endogenous corresponding gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene.

Any technique known in the art may be used to introduce a transgene under the control of the BSP regulatory region into animals to produce the founder lines of transgenic animals. Such techniques include, but are not limited to, pronuclear microinjection (Hoppe & Wagner, 1989, U.S. Pat. No. 4,873,191); nuclear transfer into enucleated oocytes of nuclei from cultured embryonic, fetal or adult cells induced to quiescence (Campbell et al., 1996, Nature 380:64-66; Wilmut et al., Nature 385:810-813); retrovirus gene transfer into germ lines (Van der Putten et al., 1985, Proc. Natl. Acad. Sci., USA 82:6148-6152); gene targeting in embryonic stem cells (Thompson et al., 1989, Cell 65:313-321); electroporation of embryos (Lo, 1983, Mol. Cell. Biol. 31:1803-1814); and sperm-mediated gene transfer (Lavitrano et al., 1989, Cell 57:717-723; see, Gordon, 1989, Transgenic Animals, Intl. Rev. Cytol. 115:171-229).

For example, for microinjection of fertilized eggs, a linear DNA fragment (the transgene) containing the regulatory region, the reporter gene and the polyadenylation signals, is excised from the reporter gene construct. The transgene may be gel purified by methods known in the art, for example, by the electroelution method. Following electroelution of gel fragments, any traces of impurities are further removed by passing through Elutip D column (Schleicher & Schuell, Dassel, Germany).

In a preferred embodiment, the purified transgene fragment is microinjected into the male pronuclei of fertilized eggs obtained from B6 CBA females by standard methods (Hogan, 1986, Manipulating the Mouse Embryo, A Laboratory Manual. Cold Spring Harbor Laboratory Press, Cold Spring Harbor, N.Y.). Mice are analyzed transiently at several embryonic stages or by establishing founder lines that allow more detailed analysis of transgene expression throughout development and in adult animals. Transgene presence is analyzed by PCR using genomic DNA purified from placentas (transients) or tail clips (founders) according to the method of Vemet et al., Methods Enzymol. 1993;225:434-451. Preferably, the PCR reaction is carried out in a volume of 100 μl containing 1 μg of genomic DNA, in 1× reaction buffer supplemented with 0.2 mM dNTPs, 2 mM MgCl₂, 600 μM each of primer, and 2.5 units of Taq polymerase (Promega, Madison, Wis.). Each of the 30 PCR cycles consists of denaturation at 94° C. for 1 min, annealing at 54° C. for 1 min, and extension at 72° C. for 1 min. The founder mice are then mated with C57B1 partners to generate transgenic F₁ lines of mice.

5.4 Screening Assays

Compounds that interfere with the abnormal function and/or growth of tumor and tissue cells with calcification potential can provide therapies targeting defects in osteotropic-related disorders including, but not limited to, localized or disseminated osteosarcoma, lung, colon, melanoma, thyroid, brain, multiple myeloma, breast and prostate cancers, and benign conditions, such as benign prostatic hyperplasia (BPH) or arterial sclerotic conditions where calcification occurs. Such compounds may be used to interfere with the onset or the progression of osteotropic-related disorders. Compounds that stimulate or inhibit promoter activity also may be used to ameliorate symptoms of osteotropic-related disorders.

Genetically engineered cells, cell lines and/or transgenic animals containing a BSP regulatory region, or fragment thereof, operably linked to a reporter gene, can be used as systems for the screening of agents that modulate BSP regulatory region activity. Such transgenic mice provide an experimental model in vivo (or can be used as a source of primary cells or cell lines for use in vitro) which can be used to develop new methods of treating osteotropic-related disorders by targeting therapeutic and/or toxic agents to cause arrest in the progression of such disorders.

The present invention encompasses screening assays designed to identify compounds that modulate activity of the BSP regulatory region. The present invention encompasses in vitro and cell-based assays, as well as in vivo assays in transgenic animals. As described hereinbelow, compounds to be tested may include, but are not limited to, oligonucleotides, peptides, proteins, small organic or inorganic compounds, antibodies, etc.

Examples of compounds may include, but are not limited to, peptides, such as, for example, soluble peptides, including, but not limited to, Ig-tailed fusion peptides, and members of random peptide libraries; (see, e.g., Lam, et al, 1991, Nature 354:82-84; Houghten, et al., 1991, Nature 354:84-86), and combinatorial chemistry-derived molecular library made of D- and/or L- configuration amino acids, phosphopeptides (including, but not limited to members of random or partially degenerate, directed phosphopeptide libraries; see, e.g., Songyang, et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

Such compounds may further comprise compounds, in particular drugs or members of classes or families of drugs, known to ameliorate the symptoms of an osteotropic-related disorder.

Such compounds include, but are not limited to, families of antidepressants such as lithium salts, carbamazepine, valproic acid, lysergic acid diethylamide (LSD), pchlorophenylalanine, p-propyldopacetamide dithiocarbamate derivatives e.g., FLA 63; antianxiety drugs, e.g., diazepam; monoamine oxidase (MAO) inhibitors, e.g., iproniazid, clorgyline, phenelzine and isocarboxazid; biogenic amine uptake blockers, e.g., tricyclic antidepressants such as desipramine, imipramine and amitriptyline; serotonin reuptake inhibitors e.g., fluoxetine; antipsychotic drugs such as phenothiazine derivatives (e.g., chlorpromazine (thorazine) and trifluopromazine)), butyrophenones (e.g., haloperidol (Haldol)), thioxanthene derivatives (e.g., chlorprothixene), and dibenzodiazepines (e.g., clozapine); benzodiazepines; dopaminergic agonists and antagonists e.g., L-DOPA, cocaine, amphetamine, α-methyl-tyrosine, reserpine, tetrabenazine, benzotropine, pargyline; noradrenergic agonists and antagonists e.g., clonidine, phenoxybenzamine, phentolamine, tropolone; nitrovasodilators (e.g., nitroglycerine, nitroprusside as well as NO synthase enzymes); and growth factors (e.g., VEGF, FGF, angiopoetins and endostatin).

In one preferred embodiment, genetically engineered cells, cell lines or primary cultures of germ and/or somatic cells containing a mammalian BSP regulatory region operatively linked to a heterologous gene are used to develop assay systems to screen for compounds which can inhibit sequence-specific DNA-protein interactions. Such methods comprise contacting a compound to a cell that expresses a gene under the control of a BSP regulatory region, or a transcriptionally active fragment thereof, measuring the level of the gene expression or gene product activity and comparing this level to the level of gene expression or gene product activity produced by the cell in the absence of the compound, such that if the level obtained in the presence of the compound differs from that obtained in its absence, a compound capable of modulating the expression of the mammalian BSP regulatory region has been identified. Alterations in gene expression levels may be by any number of methods known to those of skill in the art e.g., by assaying for reporter gene activity, assaying cell lysates for mRNA transcripts, e.g. by Northern analysis or using other methods known in the art for assaying for gene products expressed by the cell.

In another embodiment, microdissection and transillumination can be used. These techniques offer a rapid assay for monitoring effects of putative drugs on osteotropic cells in transgenic animals containing a BSP regulatory region-driven reporter gene. In this embodiment, a test agent is delivered to the transgenic animal by any of a variety of methods. Methods of introducing a test agent may include oral, intradermal, intramuscular, intraperitoneal, intravenous, subcutaneous, intranasal and via scarification (scratching through the top layers of skin, e.g., using a bifurcated needle) or any other standard routes of drug delivery. The effect of such test compounds on the osteotropic cells can be analyzed by the microdissection and transillumination of the osteoblastic cells. If the level of reporter gene expression observed or measured in the presence of the compound differs from that obtained in its absence, a compound capable of modulating the expression of the mammalian BSP regulatory region has been identified.

In various embodiments of the invention, compounds that may be used in screens for modulators of osteotropic-related disorders include peptides, small molecules, both naturally occurring and/or synthetic (e.g., libraries of small molecules or peptides), cell-bound or soluble molecules, organic, non-protein molecules and recombinant molecules that may have BSP regulatory region binding capacity and, therefore, may be candidates for pharmaceutical agents.

Alternatively, the proteins and compounds include endogenous cellular components which interact with BSP regulatory region sequences in vivo. Cell lysates or tissue homogenates may be screened for proteins or other compounds which bind to the BSP regulatory region, or fragment thereof. Such endogenous components may provide new targets for pharmaceutical and therapeutic interventions.

In one embodiment, libraries can be screened. Many libraries are known in the art that can be used, e.g., peptide libraries, chemically synthesized libraries, recombinant (e.g., phage display libraries), and in vitro translation-based libraries. In one embodiment of the present invention, peptide libraries may be used to screen for agonists or antagonists of BSP-linked reporter expression. Diversity libraries, such as random or combinatorial peptide or non-peptide libraries can be screened for molecules that specifically modulate BSP regulatory region activity. Random peptide libraries consisting of all possible combinations of amino acids attached to a solid phase support may be used to identify peptides that are able to activate or inhibit BSP regulatory region activities (Lam, K. S. et al., 1991, Nature 354:82-84). The screening of peptide libraries may have therapeutic value in the discovery of pharmaceutical agents that stimulate or inhibit the expression of BSP regulatory regions.

Examples of chemically synthesized libraries are described in Fodor et al., 1991, Science 251:767-773; Houghten et al., 1991, Nature 354:84-86; Lam et al., 1991, Nature 354:82-84; Medynski, 1994, BioTechnology 12:709-710; Gallop et al., 1994, J. Medicinal Chemistry 37(9):1233-1251; Ohlmeyer etal., 1993, Proc. Natl. Acad. Sci. USA 90:10922-10926; Erb etal., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al, 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott and Smith, 1990, Science 249:386-390; Devlin et al., 1990, Science, 249:404-406; Christian, et al., 1992, J. Mol. Biol. 227:711-718; Lenstra, 1992, J. hnmunol. Meth. 152:149-157; Kay et al., 1993, Gene 128:59-65; and PCT Publication No. WO 94/18318 dated Aug. 18, 1994.

By way of example of non-peptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) also can be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

A specific embodiment of such an in vitro screening assay is described below. The BSP regulatory region-reporter vector is used to generate transgenic mice from which primary cultures of BSP regulatory region-reporter vector germ cells are established. About 10,000 cells per well are plated in 96-well plates in total volume of 100 μl, using medium appropriate for the cell line. Candidate inhibitors of the BSP regulatory region are added to the cells. The effect of the inhibitors of the BSP regulatory region can be determined by measuring the response of the reporter gene driven by the BSP regulatory region. This assay could easily be set up in a high-throughput screening mode for evaluation of compound libraries in a 96-well format that reduce (or increase) reporter gene activity, but which are not cytotoxic. After 6 hours of incubation, 100 μl DMEM medium +2.5% fetal bovine serum (FBS) to 1.25% final serum concentration is added to the cells, which are incubated for a total of 24 hours (18 hours more). At 24 hours, the plates are washed with PBS, blot dried, and frozen at −80° C. The plates are thawed the next day and analyzed for the presence of reporter activity.

In a preferred example of an in vivo screening assay, tumor or tissue cells with calcification potential derived from transgenic mice can be transplanted into mice with a normal or other desired phenotype (Brinster et al., 1994, Proc. Natl. Acad. Sci. USA 91:11298-302; Ogawa et al., 1997, Int. J. Dev. Biol. 41:111-12). Such mice can then be used to test the effect of compounds and other various factors on osteotropic-related disorders. In addition to the compounds and agents listed above, such mice can be used to assay factors or conditions that can be difficult to test using other methods, such as dietary effects, internal pH, temperature, etc.

Once a compound has been identified that inhibits or enhances BSP regulatory region activity, it may then be tested in an animal-based assay to determine if the compound exhibits the ability to act as a drug to ameliorate and/or prevent symptoms of an osteotropic-related disorder, including, but not limited to, localized or disseminated osteosarcoma, lung, colon, melanoma, thyroid, brain, multiple myeloma, breast and prostate cancers, and benign conditions, such as benign prostatic hyperplasia (BPH) or arterial sclerotic conditions where calcification occurs.

The assays of the present invention may be first optimized on a small scale (i.e., in test tubes), and then scaled up for high-throughput assays. The screening assays of the present invention may be performed in vitro, i.e., in test tubes, using purified components or cell lysates. The screening assays of the present invention may also be carried out in intact cells in culture and in animal models. In accordance with the present invention, test compounds which are shown to modulate the activity of the BSP regulatory region in vitro, as described herein, will further be assayed in vivo in cultured cells and animal models to determine if the test compound has the similar effects in vivo and to determine the effects of the test compound on osteotropic-related disorders.

5.5 Compositions and Methods for Therapeutic Use of BSP Regulatory Region Nucleotides

BSP regulatory regions, or transcriptionally active fragments thereof, can be used to treat and/or prevent diseases, conditions or disorders that can be ameliorated by modifying the level or the expression of BSP, or a heterologous gene linked to a BSP regulatory region, in an osteotropic-specific manner. Described herein are methods for such therapeutic treatments.

The BSP regulatory region may be used to achieve tissue specific expression in gene therapy protocols. In cases where such cells are tumor cells, the induction of a cytotoxic product by the BSP regulatory region may be used in the form of cancer gene therapy specifically targeted to tumor cells with calcification potential which contain transacting factors required for BSP expression. In this way, the BSP regulatory region may serve as a delivery route for a gene therapy approach to cancers involving tumor cells with calcification potential. Additionally, antisense, antigene or aptameric oligonucleotides may be delivered to cells using the presently described expression constructs. Ribozymes or single-stranded RNA also can be expressed in a cell to inhibit the expression of a target gene of interest. The target genes for these antisense or ribozyme molecules should be those encoding gene products that are essential for cell maintenance.

The BSP regulatory region, and transcriptionally active fragments thereof, of the present invention may be used for a wide variety of purposes, e.g., to down regulate BSP gene expression, or, alternatively, to achieve osteotropic-specific expression of heterologous coding sequences.

In one embodiment, for example, the endogenous BSP regulatory region may be targeted to specifically down-regulate expression of the BSP gene. For example, oligonucleotides complementary to the regulatory region may be designed and delivered to the cells. Such oligonucleotides may anneal to the regulatory sequence and prevent transcription activation. Alternatively, the regulatory sequence, or portions thereof, may be delivered to cells in saturating concentrations to compete for transcription factor binding. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11:155-215. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In another embodiment, a gene therapy method for ameliorating osteotropicrelated disorders is provided. BSP regulatory region sequences are introduced in the osteotropic cells and used to drive osteotropic-specific expression of drugs or toxins. The method comprises introducing a BSP regulatory region sequence operatively associated with a drug or toxin gene into the osteotropic cells.

In yet another embodiment, the invention provides a gene therapy method for treatment of cancer or other proliferative disorders. The BSP regulatory region is used to direct the expression of one or more proteins specifically in osteotropic tumor cells of a patient. Such proteins may be, for example, tumor suppressor genes, thymidine kinase (used in combination with acyclovir), toxins or proteins involved in cell killing, such as proteins involved in the apoptosis pathway.

In one embodiment, the invention provides for a therapeutic agent comprising a BSP promoter which is useful for toxic gene therapy. This method includes a eukaryotic delivery vector and a toxic gene. In the preferred embodiment, the vector is adenovirus (Ad) and the gene is thymidine kinase (TK). Thus, the therapeutic agent is represented by the formula Ad-BSP-TK, but in reality the novel concept contained herein is the BSP promoter as the driving force for osteotropic-specific expression of heterologous coding sequences.

The DNA encoding the translational or transcriptional products of interest may be engineered recombinantly into a variety of vector systems that provide for replication of the DNA in large scale for the preparation of the vectors of the invention. These vectors can be designed to contain the necessary elements for directing the transcription and/or translation of the DNA sequence taken up by the osteotropic cells.

Vectors that may be used include, but are not limited to, those derived from recombinant bacteriophage DNA, plasmid DNA or cosmid DNA. For example, plasmid vectors such as pBR322, pUC 19/18, pUC 118, 119 and the M13 mp series of vectors may be used. Bacteriophage vectors may include λgt10, λgt11, λgt18-23, λZAP/R and the EMBL series of bacteriophage vectors. Cosmid vectors that may be utilized include, but are not limited to, pJB8, pCV 103, pCV 107, pCV 108, pTM, pMCS, pNNL, pHSG274, COS202, COS203, pWE15, pWE16 and the charomid 9 series of vectors. Vectors that allow for the in vitro transcription of RNA, such as SP6 vectors, also may be used to produce large quantities of RNA that may be incorporated into viral vectors.

Alternatively, recombinant replication competent or incompetent viral vectors including, but not limited to, those derived from viruses such as herpes virus, retroviruses, vaccinia viruses, adenoviruses, adeno-associated viruses or bovine papilloma virus may be engineered. While integrating vectors may be used, non-integrating systems, which do not transmit the gene product to daughter cells for many generations, are preferred for non-disease related repair and regeneration. In this way, the gene product is expressed during the repair process, and as the gene is diluted out in progeny generations, the amount of expressed gene product is diminished.

The use of tissue specific promoters to drive therapeutic gene expression would decrease further a toxic effect of the therapeutic gene on neighboring normal cells when virus-mediated gene delivery results in the infection of the normal cells. This would be important especially in diseases where systemic administration could be utilized to deliver a therapeutic vector throughout the body, while maintaining transgene expression to a limited and specific number of cell types. Moreover, since many bone growth factors, such as TGF-P, have pleiotropic effects, numerous, harmful side effects likely would be exhibited if the growth factor genes are expressed in all cells.

The invention is also based, in part, on the fact that adenoviral vectors constructed with a BSP transcriptional regulatory sequence described herein are capable of selectively driving expression of an adenovirus gene essential for replication in a tissue specific and tumor-restrictive manner. The invention is further based, in part, on the discovery that such adenoviral vectors can be used as therapeutic agents for treating prostate cancer. Thus, due to the tissue-specificity and tumor-restrictiveness of the BSP transcriptional regulatory sequence used with the adenoviral vectors, the adenovirus can be administered in a tumor-restrictive and tissue-specific manner, with the use of a BSP transcriptional regulatory sequence which allows for tissue specific expression of the adenovirus gene essential for replication and/or heterologous nucleotide sequence. An example of such a BSP transcriptional regulatory sequence is the BSP promoter which is activated only within osteotropic cells and tissues. Thus, an adenovirus vector constructed with an essential gene under the control of an BSP transcriptional regulatory sequence can be expressed effectively and specifically in targeted tumor cells and tissues, thereby minimizing the side effects of expression of the adenovirus vector in non-osteotropic cells and tissues.

In addition, due to the tissue specificity of the BSP transcriptional regulatory sequence used with the adenoviral vectors, the viral vectors of the present invention are effective therapeutic agents not only when administered via direct application, such as by injection, but also when administered systemically to the body via intravenous administration, oral administration or the like, because gene expression will be limited and localized to specific, prostatic cell and disease tissues.

In one embodiment, the invention provides an adenovirus vector comprising an adenovirus with an essential gene under transcriptional control of a BSP transcriptional regulatory sequence. The BSP transcriptional regulatory sequence is capable of mediating gene expression specific to cells which allow a BSP transcriptional regulatory sequence to function, such as for example, tumors and other diseases and disorders involving tumor and tissue cells with calcification potential, including osteotropic-related disorders including, but not limited to, localized or disseminated osteosarcoma, other osteotropic tumors including, but not limited to, lung, colon, melanoma, thyroid, brain, multiple myeloma, and especially including, without limitation, breast and prostate cancers, and benign conditions, such as BPH or arterial sclerotic conditions.

The BSP transcriptional regulatory sequence can comprise a promoter and/or enhancer or enhancer-like sequence from a BSP gene, provided that the BSP transcriptional regulatory sequence is capable of mediating gene expression specific to cells expressing BSP. In one embodiment, a BSP transcriptional regulatory sequence comprises a promoter from a BSP gene. In one embodiment, a BSP transcriptional regulatory sequence comprises an enhancer or enhancer-like sequence from a BSP gene. In one embodiment, a BSP transcriptional regulatory sequence comprises a promoter from a BSP gene and an enhancer or enhancer-like sequence from a BSP gene. In one embodiment, the BSP transcriptional regulatory sequence is transcriptionally active in cells which allow a BSP transcriptional regulatory sequence to function, such as cells expressing BSP.

In some embodiments, the BSP transcriptional regulatory sequence is human, mouse, or rat in origin. In some embodiments, the mouse or rat BSP transcriptional regulatory sequence is capable of mediating prostate-specific gene expression in humans.

In some embodiments, the adenovirus gene under control of a BSP transcriptional regulatory sequence contributes to cytotoxicity (directly or indirectly), such as a gene essential for viral replication. In one embodiment, the adenovirus gene is an early gene. In another embodiment, the early gene is E1A. In another embodiment, the early gene is E1B. In yet another embodiment, both E1A and E1B are under transcriptional control of a BSP transcriptional regulatory sequence. In other embodiments, the adenovirus gene essential for replication is a late gene. In various embodiments, the additional late gene is L1, L2, L3, L4, or L5.

In some instances, the promoter elements may be constitutive or inducible promoters and can be used under the appropriate conditions to direct high level or regulated expression of the gene of interest. Expression of genes under the control of constitutive promoters does not require the presence of a specific substrate to induce gene expression and will occur under all conditions of cell growth. In contrast, expression of genes controlled by inducible promoters is responsive to the presence or absence of an inducing agent. For example, if a cell is stably transfected with a therapeutic, inducible transgene, its expression could be controlled over the life-time of the individual. In fact, the BSP promoter, itself, is induced by glucocorticoids and ascorbic acid.

Specific initiation signals also are required for sufficient translation of inserted protein coding sequences. These signals include the ATG initiation codon and adjacent sequences. In cases where the entire coding sequence, including the initiation codon and adjacent sequences, are inserted into the appropriate expression vectors, no additional translational control signals may be needed. However, in cases where only a portion of the coding sequence is inserted, exogenous translational control signals, including the ATG initiation codon, must be provided. Furthermore, the initiation codon must be in phase with the reading frame of the protein coding sequences to ensure translation of the entire insert. These exogenous translational control signals and initiation codons can be of a variety of origins, both natural and synthetic. The efficiency and control of expression may be enhanced by the inclusion of transcription attenuation sequences, enhancer elements, etc.

In another embodiment of the present invention there is provided a method for treating osteotropic tumors comprising delivering a therapeutic agent to the tumor. The therapeutic agent comprises a recombinant adenovirus vector (Ad) containing aBSP promoter driven toxic thymidine kinase (Tk). An additional aspect of the present invention provides a method of regulating expression of Tk with the addition of a suitable prodrug including, but not limited to, acyclovir (AcV).

The therapeutic agent containing the BSP promoter-driven toxic gene therapy, in the presence of a suitable prodrug, can be administered to osteosarcoma tumors, and prostate cancer tumors, and their metastases, and many other osteotropic tumors, including, but not limited to, colon, brain, lung, breast, multiple, myeloma, thyroid, and melanoma. The present therapeutic invention comprising Ad-BSP-TK, or other vectors containing BSP promoter-driven activity, is provided to target cancers that are osteotropic, thus possessing bone-like and bone homing capabilities and eliciting an osteoblastic or osteolytic phenotype when in association with bone tissue.

In still another embodiment, the BSP regulatory region may code for a variety of factors that promote bone repair including extracellular, cell surface and intracellular RNAs and proteins. These therapeutic constructs would be useful for, inter alia, aging and certain degenerative conditions. Examples of extracellular proteins include growth factors, cytokines, therapeutic proteins, hormones and peptide fragments of hormones, inhibitors of cytokines, peptide growth and differentiation factors, interleukins, chemokines, interferons, colony stimulating factors and angiogenic factors. Examples of such proteins include, but are not limited to, the superfamily of TGF-β molecules, including the five TGF-β isoforms and bone morphogenetic proteins (BMP), latent TGF-β binding proteins, LTBP; keratinocyte growth factor (KGF); hepatocyte growth factor (HGF); platelet derived growth factor (PDGF); insulin-like growth factor (IGF); the basic fibroblast growth factors (FGF-1, FGF-2, etc.), vascular endothelial growth factor (VEGF); Factor VIII and Factor IX; erythropoietin (EPO); tissue plasminogen activator (TPA) and activins and inhibins. Hormones which may be used in the practice of the invention include, for example, growth hormone (GH) and parathyroid hormone (PTH). Examples of extracellular proteins also include the extracellular matrix proteins such as collagen, laminin and fibronectin. Examples of cell surface proteins include the family of cell adhesion molecules (e.g., the integrins, selecting, Ig family members such as N-CAM and L1 and cadherins); cytokine signaling receptors such as the type I and type II TGF-0 receptors and the FGF receptor and non-signaling co-receptors such as betaglycan and syndecan. Examples of intracellular RNAs and proteins include the family of signal transducing kinases, cytoskeletal proteins such as talin and vinculin, cytokine binding proteins such as the family of latent TGF-0 binding proteins and nuclear trans acting proteins such as transcription factors and enhancing factors.

The method comprises introducing a BSP regulatory region sequence operatively associated with a nucleic acid encoding a therapeutic compound that promotes bone synthesis and/or repair. The tissue specificity of the BSP promoter will allow for specific expression of the therapeutic compounds in osteotropic cells of interest. The use of the BSP promoter to drive therapeutic gene expression would decrease further a toxic effect of the therapeutic gene on neighboring normal cells when virus-mediated gene delivery results in the infection of the normal cells. This would be important especially in diseases where systemic administration could be utilized to deliver a therapeutic vector throughout the body, while maintaining transgene expression to a limited and specific number of cell types. Moreover, since many therapeutic growth factors, such as TGF-β, have pleiotropic effects, numerous, harmful side effects likely would be exhibited if the growth factor genes are expressed in all cells.

In yet another embodiment, the BSP regulatory region may code for a variety of genes with immune modulatory functions, e.g. for cytokines such as interleukins 1 to 15 inclusive, especially for example IL2, IL12, gamma-interferon, tumour necrosis factor, GMCSF, and/or other genes, e.g. those mentioned in specifications WO 88/00971 (CSIRO, Australian National University: Ramshaw et al) and WO 94/16716 (Virogenetics Corp; Paoletti et al).

Also the following genes can be encoded by the BSP regulatory regions of the invention: genes for interferons alpha, beta or gamma; tumour necrosis factor; granulocyte-macrophage colony-stimulating factor (GM-CSF), granulocyte colony-stimulating factor (G-CSF), macrophage colony-stimulating factor (N-CSF), chemokines such as neutrophil activating protein NAP, macrophage chemoattractant and activating factor MCAF, RANTES, macrophage inflammatory peptides MIP-1a and MIP-1b, complement components and their receptors, accessory molecules such as 87.1, 87.2, ICAM-1.2 or 3 or cytokine receptors. Where nucleotide sequences encoding more than one immunomodulating protein are inserted, they may comprise more than one cytokine or may represent a combination of cytokine and accessory molecule(s).

5.5.1 Modulatory Antisense, Ribozyme and Triple Helix Approaches

In another embodiment, the types of conditions, disorders, or diseases involving tumor and tissue cells with calcification potential which may be prevented, delayed, or rescued by modulating osteotropic-specifc gene expression by using a BSP regulatory region in conjunction with well-known antisense, gene “knock-out,” ribozyme and/or triple helix methods, are described. Such molecules may be designed to modulate, reduce or inhibit either unimpaired, or if appropriate, mutant osteotropic gene activity. Techniques for the production and use of such molecules are well known to those of skill in the art.

Antisense RNA and DNA molecules act to directly block the translation of mRNA by hybridizing to targeted mRNA and preventing protein translation. Antisense approaches involve the design of oligonucleotides which are complementary to an mRNA sequence. The antisense oligonucleotides will bind to the complementary mRNA sequence transcripts and prevent translation. Absolute complementarity, although preferred, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, oligonucleotides complementary to non-coding regions of the sequence of interest could be used in an antisense approach to inhibit translation of endogenous mRNA. Antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit sequence expression. It is preferred that these studies utilize controls that distinguish between antisense gene inhibition and nonspecific biological effects of oligonucleotides. Additionally, it is envisioned that results obtained using the antisense oligonucleotide are compared with those obtained using a control oligonucleotide. It is preferred that the control oligonucleotide is of approximately the same length as the test oligonucleotide and that the nucleic acid of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger, et al., 1989, Proc. Natl. Acad. Sci. U.S.A. 86:6553-6556; Lemaitre, et al., 1987, Proc. Natl. Acad. Sci. U.S.A. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6:958-976) or intercalating agents (see, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxyrnethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier, et al., 1987, Nucl. Acids Res. 15:6625-6641). The oligonucleotide is a 2′-0-methylribonucleotide (Inoue, et al., 1987, Nucl. Acids Res. 15:6131-6148), or a chimeric RNA-DNA analogue (Inoue, et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein, et al. (1988, NucL Acids Res. 16:3209), methylphosphonate oligonucleotides can be prepared by use of controlled pore glass polymer supports (Sarin, et al., 1988, Proc. Natl. Acad. Sci. U.S.A. 85:7448-7451), etc.

While antisense nucleotides complementary to the osteotropic specific coding region sequence could be used, those complementary to the transcribed, untranslated region are most preferred.

Antisense molecules should be delivered to cells that express the osteotropic sequence in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies which specifically bind receptors or antigens expressed on the target cell surface) can be administered systemically.

A preferred approach to achieve intracellular concentrations of the antisense sufficient to suppress translation of endogenous mRNAs utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs which will form complementary base pairs with the endogenous sequence transcripts and thereby prevent translation of the mRNA. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bemoist and Chambon, 1981, Nature 290:304-310), the promoter contained in the 3′-long terminal repeat of Rous sarcoma virus (Yamamoto, et al., 1980, Cell 22:787-797), the herpes thymidine kinase promoter (Wagner, et al., 1981, Proc. Natl. Acad. Sci. U.S.A. 78:1441-1445), the regulatory sequences of the metallothionein gene (Brinster, et al., 1982, Nature 296:39-42), etc. Any type of plasmid, cosmid, YAC or viral vector can be used to prepare the recombinant DNA construct that can be introduced directly into the tissue site. Alternatively, viral vectors can be used that selectively infect the desired tissue, in which case administration may be accomplished by another route (e.g., systemically).

Ribozyme molecules designed to catalytically cleave target gene mRNA transcripts can also be used to prevent translation of target gene mRNA and, therefore, expression of target gene product. (See, e.g., PCT International Publication WO90/11364, published Oct. 4, 1990; Sarver, et al, 1990, Science 247, 1222-1225).

Ribozymes are enzymatic RNA molecules capable of catalyzing the specific cleavage of RNA. (For a review, see Rossi, 1994, Current Biology 4:469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

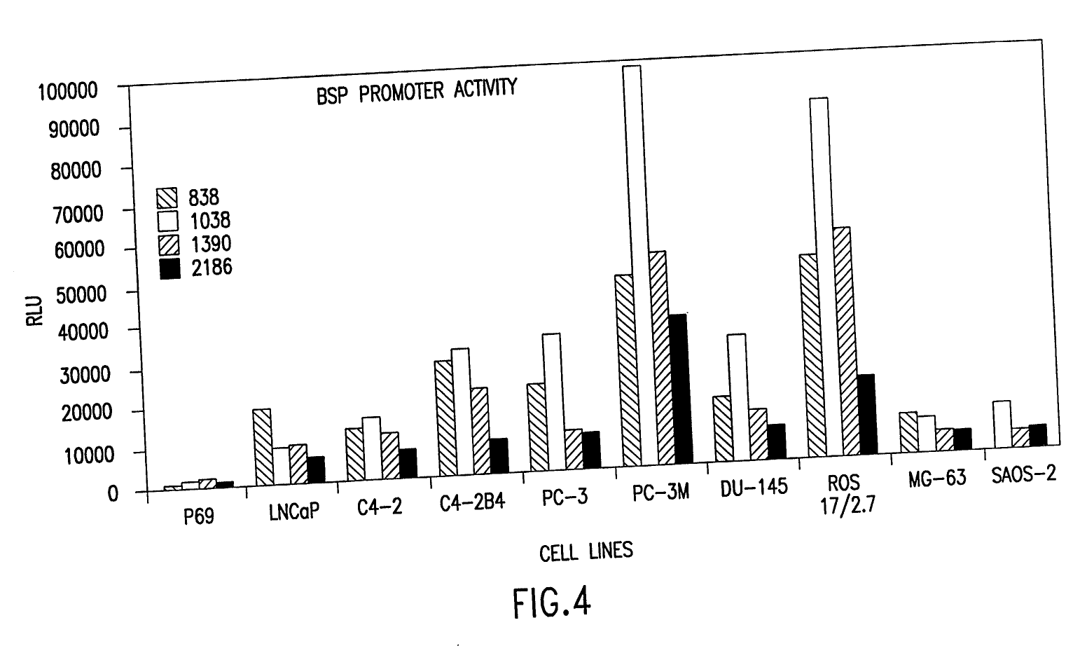

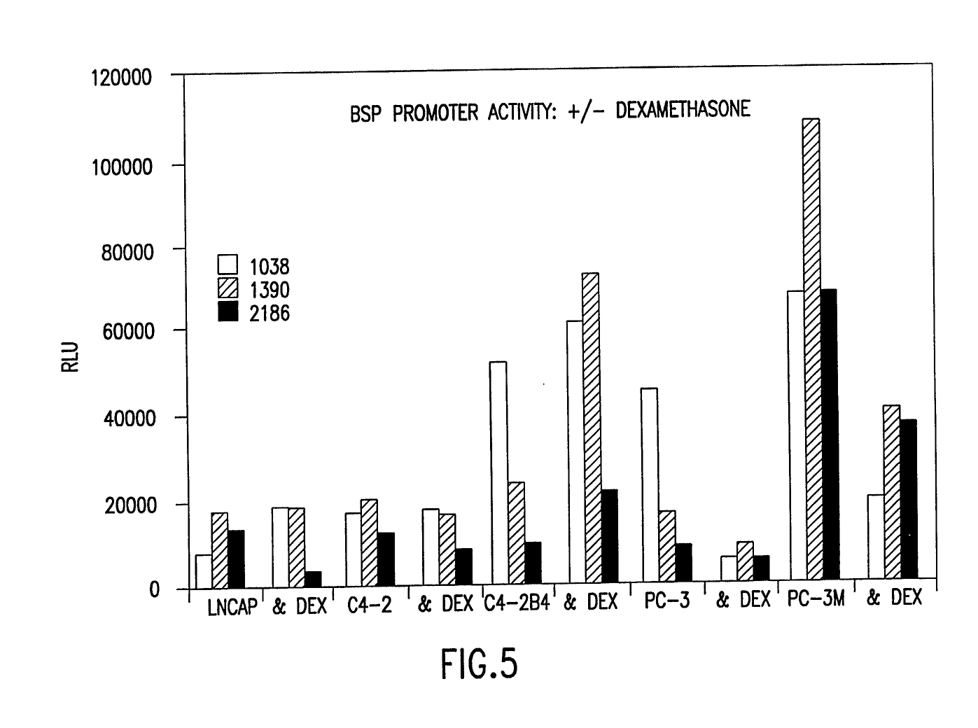

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions which form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334:585-591, which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been and Cech, 1986, Cell, 47:207-216). The Cech-type ribozymes have an eight base pair active site that hybridizes to a target RNA sequence whereafter cleavage of the target RNA takes place. The invention encompasses those Cech-type ribozymes that target eight base-pair active site sequences that are present in the target gene.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes, unlike antisense molecules, are catalytic, a lower intracellular concentration is required for efficiency.

Endogenous target gene expression can also be reduced by inactivating or “knocking out” the target gene or its promoter using targeted homologous recombination (e.g., see Smithies, et al, 1985, Nature 317:230-234; Thomas and Capecchi, 1987, Cell 51:503-512; Thompson, et al., 1989, Cell 5:313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells which express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures which prevent transcription of the target gene in target cells in the body. (See generally, Helene, 1991, Anticancer Drug Des., 6(6):569-584; Helene, et al., 1992, Ann. N.Y. Acad. Sci., 660:27-36; and Maher, 1992, Bioassays 14(12):807-815).

Nucleic acid molecules to be used in triple helix formation for the inhibition of transcription should be single stranded and composed of deoxynucleotides. The base composition of these oligonucleotides must be designed to promote triple helix formation via Hoogsteen base pairing rules, which generally require sizable stretches of either purines or pyrimidines to be present on one strand of a duplex. Nucleic acids may be pyrimidine-based, which will result in TAT and CGC+triplets across the three associated strands of the resulting triple helix. The pyrimidine-rich molecules provide base complementarity to a purine-rich region of a single strand of the duplex in a parallel orientation to that strand. In addition, nucleic acid molecules may be chosen which are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles which the possibility may arise wherein the concentration of normal target gene product present may be lower than is necessary for a normal phenotype. In such cases, to ensure that substantially normal levels of target gene activity are maintained, therefore, nucleic acid molecules which encode and express target gene polypeptides exhibiting normal target gene activity may be introduced into cells via gene therapy methods such as those described, below, in Section 5.4.2 which do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Anti-sense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid-phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

5.5.2 Gene Replacement Therapy

The nucleic acid sequences of the invention, described above, can be utilized for transferring recombinant nucleic acid sequences to cells and expressing said sequences in recipient cells. Such techniques can be used, for example, in marking cells or for the treatment of a disorder involving tumor or tissue cells with calcification potential. Such treatment can be in the form of gene replacement therapy. Specifically, one or more copies of a normal gene or a portion of the gene that directs the production of a gene product exhibiting normal gene function, may be inserted into the appropriate cells within a patient, using vectors that include, but are not limited to adenovirus, adeno-associated virus and retrovirus vectors, in addition to other particles that introduce DNA into cells, such as liposomes.

Methods for introducing genes for expression in mammalian cells are well known in the field. Generally, for such gene therapy methods, the nucleic acid is directly administered in vivo into a target cell or a transgenic mouse that expresses a BSP regulatory region operably linked to a heterologous coding sequence. This can be accomplished by any method known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), by direct injection of naked DNA, by use of microparticle bombardment (e.g., a gene gun; Biolistic, Dupont), by coating with lipids or cell-surface receptors or transfecting agents, by encapsulation in liposomes, microparticles, or microcapsules, by administering it in linkage to a peptide which is known to enter the nucleus or by administering it in linkage to a ligand subject to receptor-mediated endocytosis (see e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992; WO 92/22635 dated Dec. 23, 1992; WO92/20316 dated Nov. 26, 1992; WO93/14188 dated Jul. 22, 1993; WO 93/20221 dated Oct. 14, 1993). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In one embodiment, techniques for delivery involve direct administration, e.g., by stereotactic delivery of such gene sequences to the site of the cells in which the gene sequences are to be expressed.

Additional methods that may be utilized to increase the overall level of gene expression and/or gene product activity include using targeted homologous recombination methods, as discussed above, to modify the expression characteristics of an endogenous gene in a cell or microorganism by inserting a heterologous DNA regulatory element such that the inserted regulatory element is operatively linked with the endogenous gene in question. Targeted homologous recombination can thus be used to activate transcription of an endogenous gene that is “transcriptionally silent”, i.e., is not normally expressed or is normally expressed at very low levels, or to enhance the expression of an endogenous gene that is normally expressed.

Further, the overall level of target gene expression and/or gene product activity may be increased by the introduction of appropriate target gene-expressing cells, preferably autologous cells, into a patient at positions and in numbers that are sufficient to ameliorate the symptoms of an osteotropic-related disorder. Such cells may be either recombinant or non-recombinant.

When the cells to be administered are non-autologous cells, they can be administered using well known techniques that prevent a host immune response against the introduced cells from developing. For example, the cells may be introduced in an encapsulated form which, while allowing for an exchange of components with the immediate extracellular environment, does not allow the introduced cells to be recognized by the host immune system.

Additionally, compounds, such as those identified via techniques such as those described above that are capable of modulating activity of a BSP regulatory region can be administered using standard techniques that are well known to those of skill in the art.

5.6 Pharmaceutical Preparations and Methods of Administration

The compounds that are determined to modify BSP regulatory region activity or gene product activity can be administered to a patient at therapeutically effective doses to treat or ameliorate a disorder involving tumor or tissue cells with calcification potential. A therapeutically effective dose refers to that amount of the compound sufficient to result in amelioration of symptoms of such a disorder.

5.6.1 Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

5.6.2 Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In certain embodiments, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

For topical application, the compounds may be combined with a carrier so that an effective dosage is delivered, based on the desired activity.

In addition to the formulations described previously, the compounds also may be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### 6 EXAMPLE

### Construction of a Recombinant Adenoviral Vector Containing the BSP Promoter Upstream of Thymidine Kinase

6.1 Materials and Methods

6.1.1 Cells and Cell Culture

ROS 17/2.8, a rat osteoblastic osteosarcoma cell line, which is obtained by harvesting cells from tumor explants grown in culture (ROS 17/2.8 was obtained from the University of Texas Dental Branch, Houston, Tex.). MG-63, a human osteoblast-derived osteosarcoma cell line; 293, a transformed human embryonic kidney cell line; and NItI 3T3, an embryonic mouse fibroblast cell line, were purchased from American Type Culture Collection (Rockville, Md.). WH, a human bladder transitional cell carcinoma, was established by our laboratory (described in Gleave, M. E., Haich, J. T., Wu, H. C., Hong, S. J. Zhau, H. F., Guthrie, P. D., and Chung, L. W. K. Epidermal growth factor receptor-mediated autocrine and paracrine stimulation of human transitional cell carcinoma. Cancer Res., 53:5300-5307, 1993). The ROS 17/2.8 and MG-63 cell lines are considered the osteoblast lineage because of their morphological, biochemical, and molecular characteristics, they were incubated in DMEM (Life Technologies, Inc., Grand Island, N.Y.) and 20% F12K (trying Scientific, Santa Ana, Calif.) supplemented with 100 units/ml penicillin, 100 μl/ml streptomycin, and 10% FB5 (Sigma Chemical Colo., St. Louis, Mo.). The WH and NIH 3T3 cell lines were maintained in T medium, (as described inKo, S-C., Gotoh, A., Thalmann, G. N., Zhdu, II. F., Johnston, D. A., Zhang, W. W., Kao, C., and Chung, L. W. K. Molecular therapy with recombinant p53 adenovirus in an androgen independent, metastatic human prostate cancer model. Hum. Gene Ther., 7:1683-1691, 1996), containing 5% FBS (fetal bovine serum). The 293 cells were maintained in MEM (Life Technologies, Inc.) with 10% FBS and 1% tryptose phosphate broth (Life Technologies, Inc.). The cells were fed three times a week with fresh gmwth medium unless otherwtse indicated.

6.1.2 Results

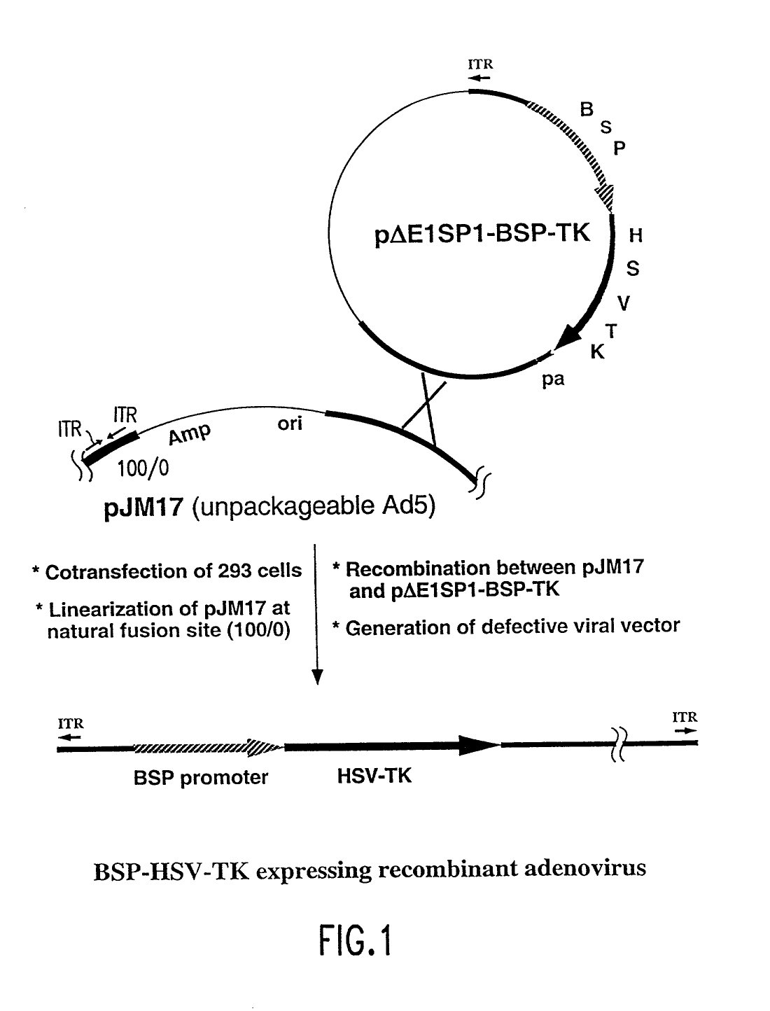

Construction of the recombinant Ad-BSP-TK virus was accomplished as shown in FIG. 1. All plasmids were constructed according to standard protocols. Briefly, pΔE1SP1, a shuttle vector which contains the 5′ end part of the adenovirus genome with the El-region deleted, was digested witb Xho-1 (New England Biolabs, Beverly, Mass.) and treated with alkaline phosphatase (Boehringer Mannheim Biochemicals, Indianapolis, Ind.) according to the supplier's protocols. The 1418 bp BSP fragment was prepared using PCR. Specifically, a first primer set was used to amplify a 1467 bp insert. These primers were:

BSP1-GTGGCACATATACACCATGG

BSP2-AATCTTACCCTCTGGCAGTC.

Internal primers to the 1467 bp fragment were then used to generate the 1418 bp BSP promoter. The internal primers were:

BSP3-CCATGGAATACTATGCAGCC

BSP4-TGGAGTGAGGAAGCAGGCTC.

The PCR-generated BSP promoter fragment was then ligated into the pΔE1SP1 Ad vector using T4 ligase (New England Biolabs). An expression clone pΔE1SPISPI-BSP-TK and pJM17, a circular adenovirus genome (with E1 region deleted and replaced with PBR322 DNA), is cotransfected in 293 cells. The recombinant vector is then purified from adenovirus infected cells, into 293 cells by the N-[1-(2,3-dioleoyloxyl)propyl]-N,N,N-trimethylammoniummethyl sulfate (Boehringer Mannheim Biochemicals)-mediated transfection metbod, Zhang, W-W., Fang, X., Branch, C. D., Mazur, W., French, B, A., and Roth, J. A. Generation and identification of recombinant adenovirus by liposome-mediated transfection and PCR analysis, Biotechniques, 15:868-872, 1993. The culture medium of the 293 cells showing the completed cytopathic effect was collected and centrifuged at 1000×g for 10 minutes. The pooled supernatants were aliquoted and stored at −80° C. as primary viral stock. Viral stocks were propagated in 293 cells, and selected clones of Ad-BSP-TK virus were obtained by plaque purification according to the method of Graham and Prevec, Graham, F. L., and Prevec, L. Manipulation of adenovirus vectors, Vol. 7, pp. 109128. Clifton, N. J.: The Humana Press, Inc., 1991. One of the viral clones was propagated in 293 cells; cells were harvested 36 to 40 hours after infection, pelle ted, resuspended in PBS, and lysed. Cell debris was removed by subjecting the cells to centrifugation, and the virus in the cell lysate was purified by CsCl₂ gradient centrifugation. Concentrated virus was dialyzed, aliquoted, and stored at −80° C. The viral titer was determined hy plaque assay. The control viruses used in this study, Ad-RSV-β-Gal and Ad-CM V-β-Gal, were constructed in a similar manner (described in Ko, S-C., Gotoh, A., Thalmann, G. N., Zhou, H. E., Johnston, D. A., Zhang, W. W., Kao C., and Chung, L. W. IC. Molecular therapy with recombinant p53 adenovirus in an androgen independent, metastatic human prostate cancer model. Hum. Gene Ther., 7:1683-1691, 1996).

### 7 EXAMPLE

### Determination of BSP mRNA in Various Cell Lines.

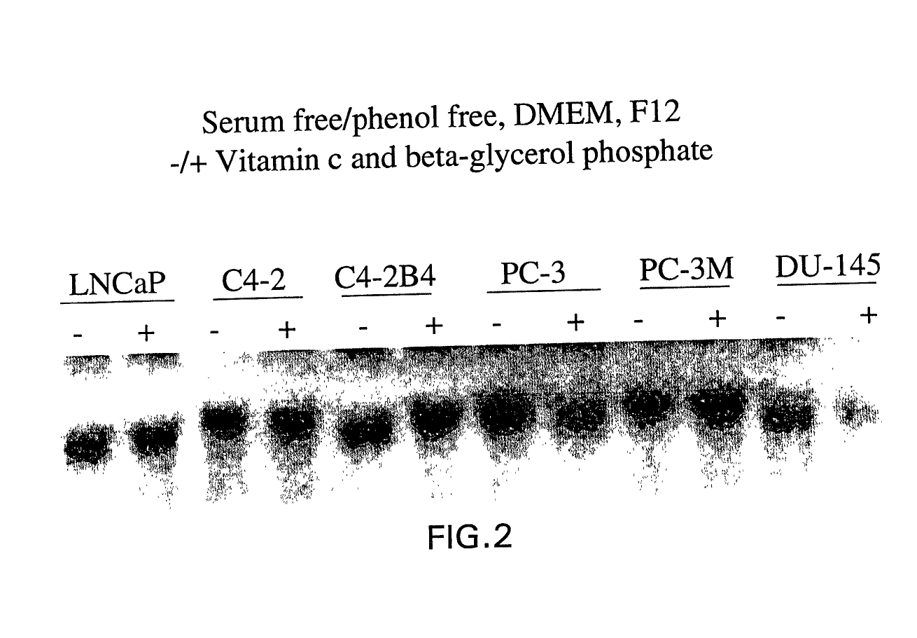

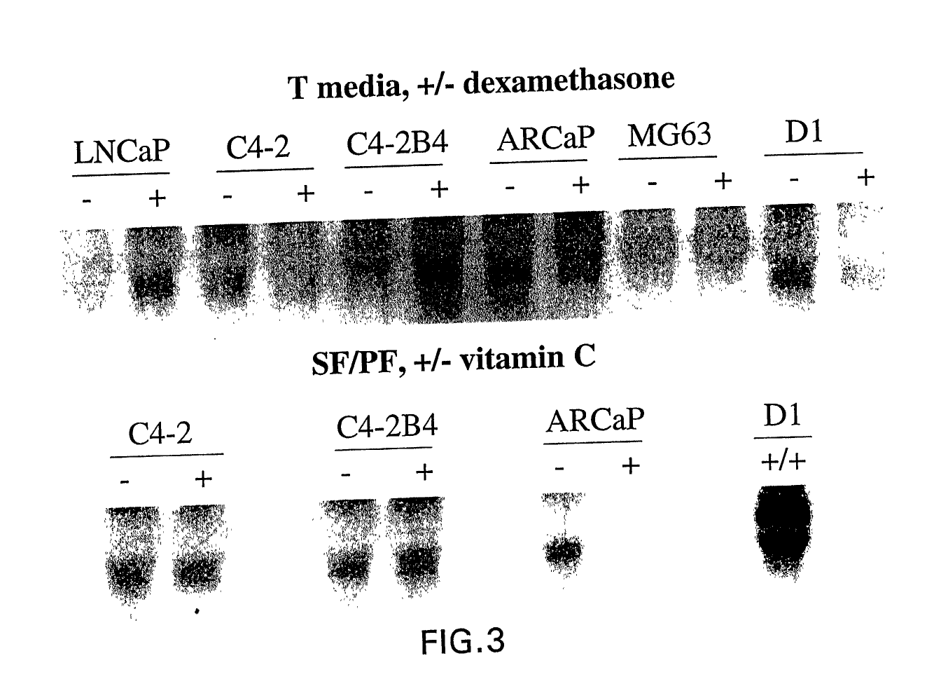

Northern Blots were performed according to standard procedures. For exemplary conditions, see, “Molecular Cloning: A Laboratory Manual”, 2nd ed., Cold Spring Harbor Laboratory Press, Sambrook, J., E. F. Fritsch and T. Maniatis eds., 1989. Cells were grown in serum free, phenol free growth media (DMEM, F12) with or without L-ascorbic acid (vitamin C) and a phosphate donor, beta-glycerol phosphate. (FIG. 2) The banding pattern seen at −1.6 kilodalton represents the expression of BSP mRNA in all prostate cell lines tested, including androgen dependent (LNCaP) and androgen-independent types (C4-2, C4-2 B4, PC-3, PC-3M, DU- 145). Androgen dependency is defined here as the ability of tumors to be formed in either intact (androgen independent) or in castrated male (androgen independent) hosts. In addition, the expression appears greater in C4-2B4, a cell type with greatest affinity to bone. Under this “mineralizing” (vitamin C and phosphate) condition, the C4-2B4 expression is upregulated. FIG. 3 shows BSP expression (on the top) in the prostate cancer cells (LNCaP, C4-2, C4-2B4 and ARCaP), the human osteosarcoma cell line (MG63), and the mouse bone marrow cell line (D1). Indeed, expression is higher in the prostate cancer lines than the sarcoma or bone cells. Once again the C42B4 cells, with the highest tropism to bone, appears to have an increased expression with the addition of dexamethasone in the media. Dexamethasone is known to increase expression of BSP, both transcriptionally and post-transcriptionally. On the bottom of FIG. 3, once again, in a separate experiment, BSP is present in all the human prostate cell lines (C4-2, C4-2B4, ARCaP) which are all known to be highly metastatic human prostate cancer cell lines, and the mouse bone marrow cells (D1).

### 8 EXAMPLE

### BSP Promoter Activity

8.1 Materials and Methods

Cells (300,00/well) were exposed to a BSP promoter-luciferase vector (2.5 ug) with liposome mediated transfection for 4-6 hours, then allowed to grow approximately 2 days at which time cell lysates were obtained and assayed for relative luciferase activity (RLU). All values were standardized with cotransfection of CMV-betagalactosidase reporter plasmid (0.5 ug).

8.2 Results

In FIG. 4, the bars indicate 3 to 4 separate experiments run in at least duplicate for four separate BSP promoter constructs with promoter lengths varied from 800 to 2200 base pairs (838 base pairs (bp), 1038 bp, 1390 bp, and 2186 bp). The cell line P69 is considered a “normal” prostate cell type without any tumoregenic or metastatic ability, and its BSP promoter-driven activity is very low. There is increasing activity in the lineage related cell sublines LNCaP, C4-2, C4-2B4 with increasing tumorogenic, metastatic and bone homing tropism. Likewise there is increasing activity in the PC-3 to PC-3M progression model sublines. The increased BSP promoter activity evidenced here as the tumor cells become more invasive and metastatic displays the truly tumor-specific activity of this promoter, especially as one recognizes the virtual absence of BSP promoter driven activity of the “normal” P69 cell type. In addition, another androgen independent cell line DU-145 has substantial activity. Notice the known rat BSP expressing osteosarcoma cell line ROS 17/2.7 also has very high activity. The human osteosarcoma cell lines MG-63 and SAOS-2 also have significant expression (>10,000 RLU). The levels of expression of therapeutic genes in the prostate and osteosarcoma tumors will parallel the BSP activity witnessed here.

In FIG. 5, BSP promoter activity in vitro under baseline (T media, 5% PBS) conditions and with an exogenous glucocorticoid, dexamethasone, is assayed. The figure once again portrays luciferase activity and the cell lines with three different length promoter constructs (1038,1390 and 2186 bp). The osteoblastic C4-2B4 cell sub-line appears to be stimulated by the glucocorticoid. The osteolytic PC-3 and PC-3M cell lines have decreased activity under the increased glucocorticoid environment.

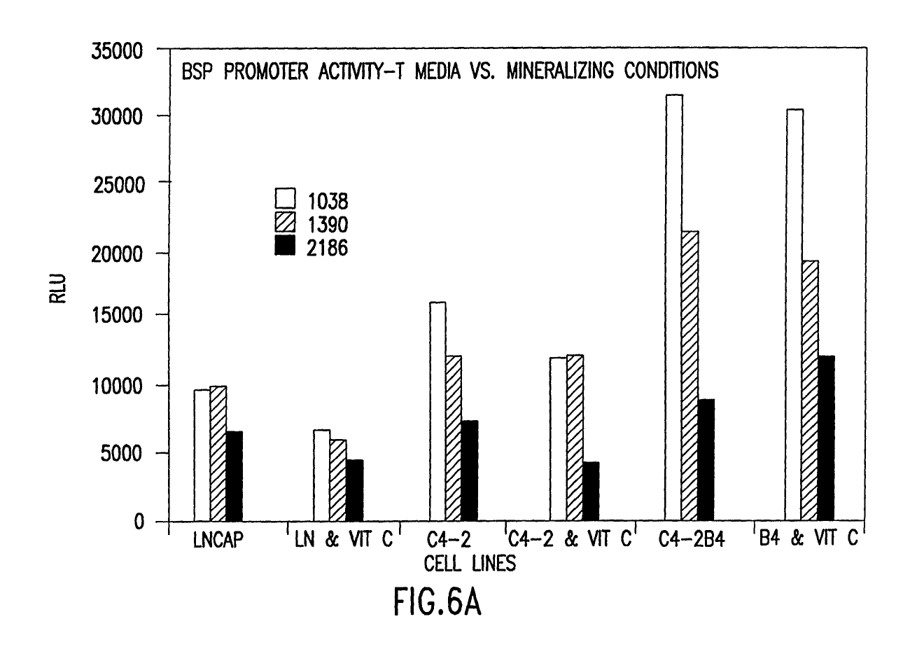

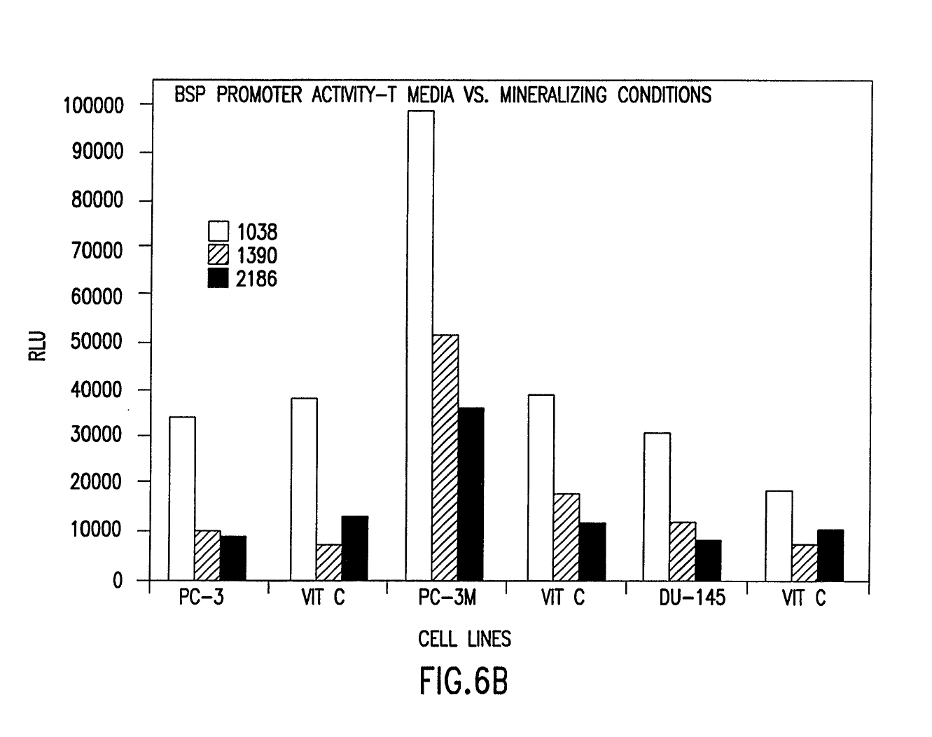

FIG. 6 shows BSP promoter expression as RLU in various prostate cancer cell lines under standard conditions (T media) and under mineralization conditions (with Lascorbic acid). It should be noted that the expression of the longest construct for BSP promoter, 2186 base pairs, elicits a hightened response in the C4-2B4, PC-3, and Du-145 cells under the L-ascorbic acid conditions. Thus, it is expected that in patients with similar tumor cell phenotypes, the expression of longer BSP promoter-driven therapeutic genes can be augmented with the concurrent exogenous administration of L-ascorbic acid (vitamin C).

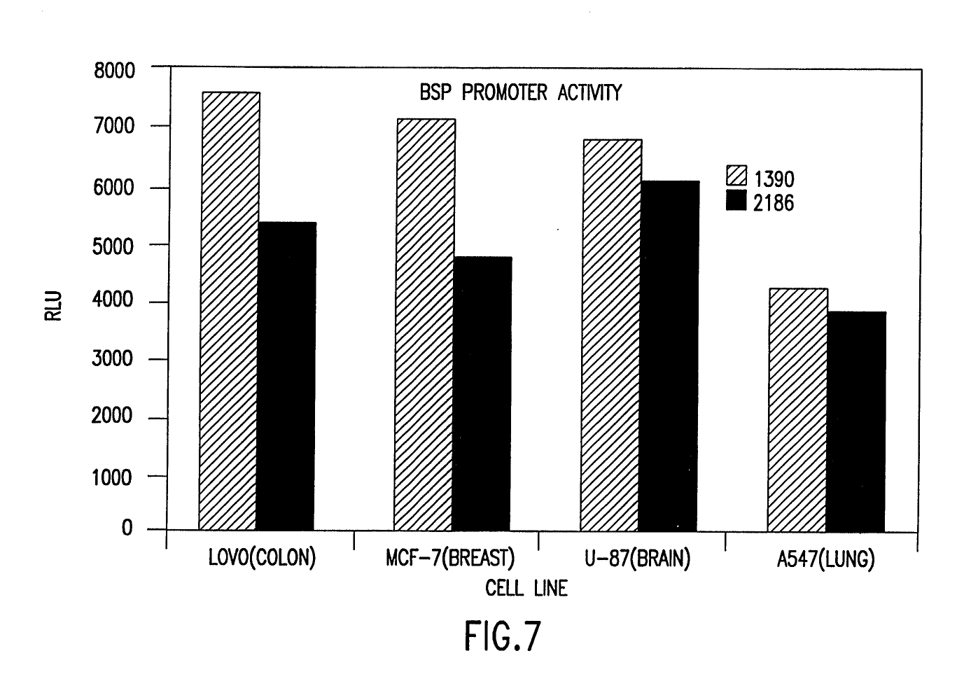

FIG. 7 shows BSP promoter-driven luciferase activity in other cancer cell types. This experiment was performed in precisely the same manner as for FIG. 6. The BSP promoter constructs used in these tumors were the 1390 and 2186 base pair lengths. The data reveals activity in colon cancer (Lovo), breast cancer (MCF-7), brain cancer (U-87, a glioblastoma multiform type), and lung cancer (A547) cell lines. The level of activity is not as high as that seen in prostate cancer and osteosarcoma, but it is substantially above (48×) the baseline level witnessed in the normal P69 cell type. Thus, the BSP promoter can drive expression of toxic genes in these cancer cell types also, and thus, be used in patients with these tumors.

### 9 EXAMPLE

### In Vitro Cytotoxicity Assays with Ad-BSP-TK

Cells were grown in T-media and infected with virus on day zero. The assay was performed over six days. Each group of cells was exposed to either (a) nothing (control), (b) ACV, (c) virus (Ad-BSP-TK) or (d) virus plus ACV.

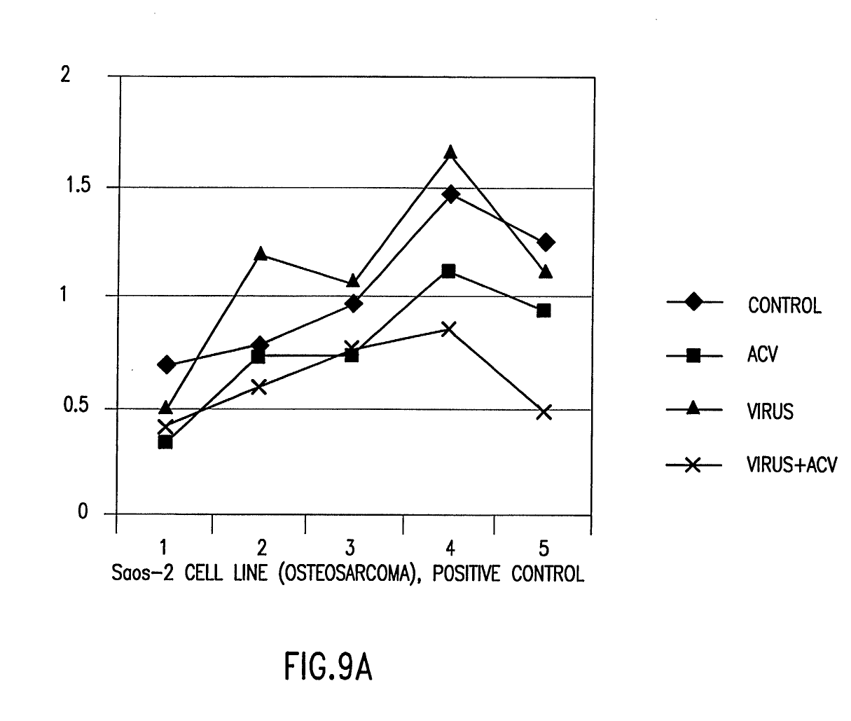

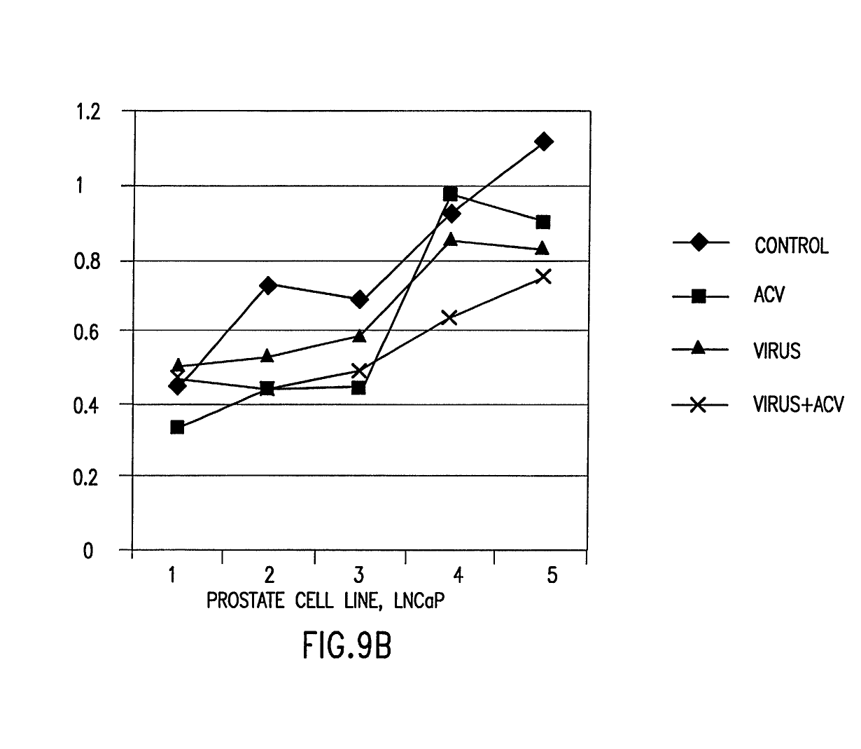

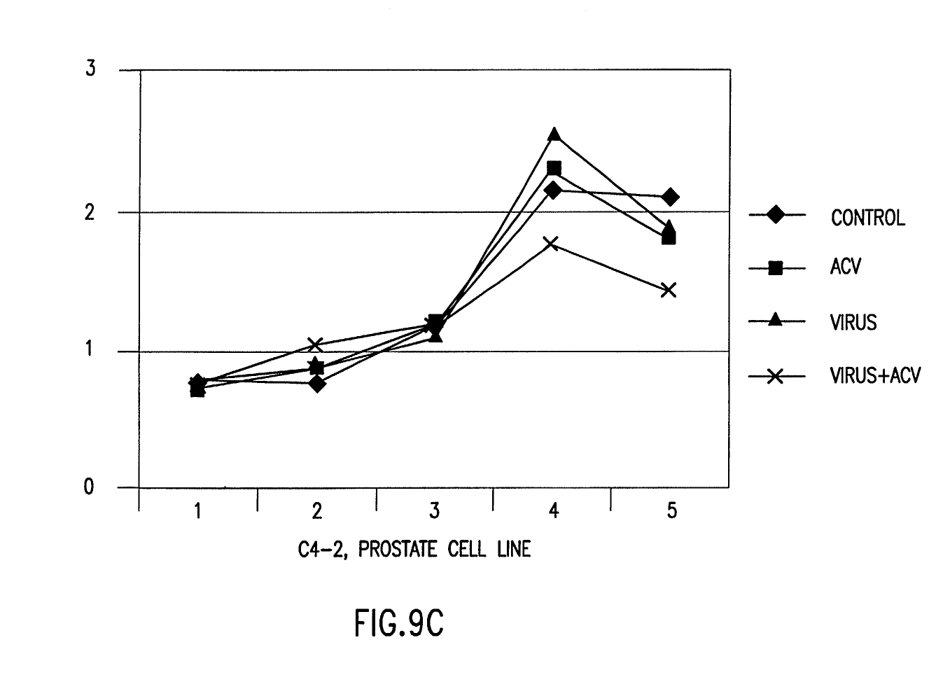

FIG. 9A, which provides data regarding the osteosarcoma cell line Saos-2, is the positive control because it produces BSP. The figure shows that virus plus ACV cells did not proliferate as well as the cells in the other three groups. FIG. 9B shows the results using the prostate cell line LNCaP. Similar to the results with Saos-2, the virus plus ACV treated cells did not proliferate as much as the cells in the control groups. FIG. 9C shows the results using the prostate cell line C4-2. Similar to the previous results, the virus plus ACV treated cells did not proliferate as much as the cells in the control groups.

### 10 EXAMPLE

### Inhibition of Tumor Growth In Vivo

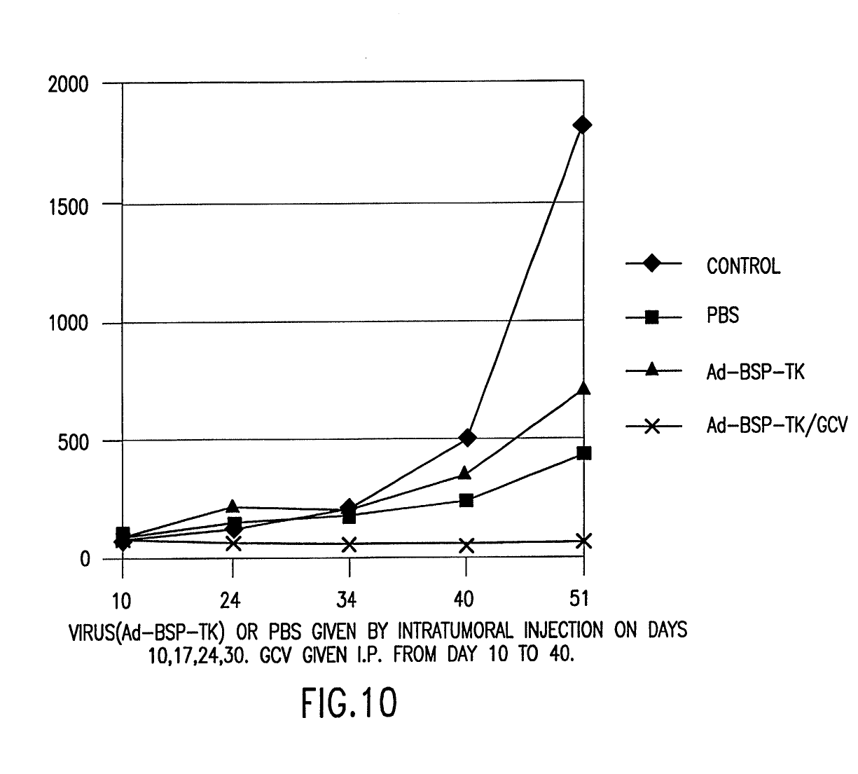

FIG. 10 represents an in vivo experiment performed with 16 animals. Briefly, congenitally athymic nude (nu/nu) mice (Harlan Co., Houston, Tex.) 5 to 6 weeks of age, were inoculated s.c. with 2 million PC3 prostate cancer cells. When the tumor became palpable (4-5 mm in diameter), approximately 10 days after cell injection, the animals were randomly assigned to four experimental groups: group: 1, control group with no treatment; 2, intratumoral PBS on days 10, 17, 24 and 30; 3, Ad-BSP-TK only; and 4, Ad-BSP-TK plus GCV given on days 10-40. For Ad-BSP-TK injections, a microliter syringe fitted with a 28 gauge needle was used to deliver 75 μl of Ad-BSP-TK (1×10⁹ plaque-forming units). The Ad-BSP-TK was injected intratumorally along both the long and short axes of the tumor; one injection parallel to the long axis, and one perpendicular to the axis. The needle point was then rotated within the tumor to maximize the area of Ad delivery. Tumor volume was calculated by the following formula: volume (a rotational ellipsoid)-M₁xM₂²×0.5236 (M₁, long axis; M₂, short axis; Ko. S-C., Gotoh, A., Thalmann, G. N., Zhou, H. E., Johnston, D. A, Zhang, W. W., Kao, C., and Chung, L. W. K. Molecular therapy with recombinant p53 adenovirus in an androgen independent, metastatic human prostate cancer model. Hum. Gene Ther., 7:1683-1691, 1996). Gancyclovir (GCV) treatment only or Ad-BSP-TK plus GCV experimental groups were treated with an i.p. injection of GCV at a dose of 40 mg/kg body weight. AD-BSP-Tk and/or GCV treatment did not adversely affect the body weight of experimental animals.

The data demonstrate that the virus plus prodrug treated group had minimal tumor growth over the 51 day period of the experiment while the three control groups had quite a large increase in tumor growth. Thus, the data demonstrate that the Ad-BSP-TK viral vector is effective at ameliorating and/or treating tumors with calcification potential in vivO.

10.1 Discussion

The present invention unexpectedly demonstrates that BSP promoter-driven genes, when infected into susceptible osteotropic tumors and cells of osteoblastic lineage, such as osteosarcomas (ROS, MG63, Saos-2), prostate (LNCaP, C4-2, C4-2B4 PC-3 PC3M, Du-145, ARCaP), colon (Lovo), lung (A547), brain (U-87), and breast (MCF-7) express very high levels of reporter gene, and thus are expected also to efficiently express high levels of chosen toxic or therapeutic gene when in combination with known delivery vector, including, but not limited to, Ad-BSP-TK. Further, the invention demonstrates that addition of a suitable prodrug, such as AcV, when combined with Ad-BSP-TK system, will effect the cessation, inhibition and cytotoxicity of said osteotropic tumors and their associated osteoblastic supporting stroma. Since BSP is primarily expressed in fully differentiated osteoblasts or tissues or tumors with the capability to mineralize, and this BSP promoter can drive efficient expression of chosen genes, either reporter, therapeutic or toxic, in osteotropic tumors as evidenced herein, the present invention unexpectedly teaches that BSP promoter constructs constitute an essential tumor-specific gene therapy that can inhibit the growth of certain osteotropic tumors (osteosarcoma, prostate, etc.) while sparing the surrounding normal tissues, or non-osteoblastic or non-osteolytic lineage cells of significant damage, and when applied systemically can prevent destruction of inappropriate tissue, while still obtaining the desired destructive effect on tumor growth. Further, the BSP promoter-driven therapeutic gene therapy is superior to conventional gene therapies for osteotropic tumors when the therapeutic gene expression is driven by universal promoters such as cytomegalovirus (CMV) and the long terminal repeat promoters of Rous Sarcoma Virus (RSV), because these universal promoters cannot distinguish the specific targeted tumor, and thus may cause inappropriate damage to nonselected tissues by expression of toxic gene in normal cells.

As mentioned previously, one therapeutic application of BSP promoterdriven gene therapy is to target cancers having the ability to metastasize to the skeleton. One mechanism for malignant cell recruitment to bone is that the osteotroblastic cells may synthesize and secrete products that are able to stimulate the growth, adhesion, and migration of osteotropic tumors such as prostate and breast cancer, and thus the reciprocal relationship between foreign tumor epithelium (cancer) and supporting bone stroma provides a favorable environment (soil) for accelerated tumor growth. The proliferation and migration of prostate or breast tumor cells may also themselves secrete paracrine growth factors that stimulate osteoblast or osteoclast bone cell growth at sites of bone metastases that results in induction of predominantly osteoblastic (e.g. prostate) or osteolytic (e.g. breast) phenotypes in the skeleton of afflicted patients. Since tumor cell growth is intimately affected by the surrounding stroma and these reciprocal interactions exist between certain tumors (prostate, osteosarcoma, breast, etc.) and bone stroma (osteoblast, or osteoclast), the development of BSP promoter-based gene therapy to target tumors, their metastasis and the supporting stroma represents a new and highly specific gene therapy modality for patients afflicted with these tumors. Indeed, BSP expression is distinct even from the other truly osteoblast specific gene, osteocalcin. Thus, BSP driven toxic gene therapy, most commonly in the form of Ad-BSP-TK, exhibits various therapeutic implications: a) BSP promoter driven gene therapy (Ad-BSP-TK) affects the expression of toxic compounds to osteosarcoma and prostate cancer cells and also eradicates osteoblastic cells that may be required to maintain the survival of osseous metastatic deposits of certain osteotropic tumors. Moreover, although the therapy may eradicate the growth of some normal osteoblastic cells, this is not a concern since the expression of BSP is highly specific and should not unduly harm the host by expression of toxic compounds in a wide variety of tissues; b) BSP promoter driven gene application can express high levels of therapeutic target genes in many calcified tumors, and is a reasonable choice for eradicating these tumors primary focus and its metastatic deposits in conjunction with the supporting stroma; c) BSP promoter-driven therapeutic gene treatments in conjunction with appropriate vehicles may be used in conjunction with conventional chemotherapy, surgery or radiation techniques or other novel therapies in reducing tumor burden and associated morbidity in local and metastatic deposits associated with various susceptible osteotropic human or eukaryotic tumors; d) Long lasting anti-tumor immunity might be elected against the remaining osteoblastic cells and tumor cells from BSP promoter-driven killing of tumor cells; e) BSP-promoter driven constructs may be used for the delivery and expression of therapeutic genes for the treatment of benign diseases such as BPH, arterial sclerosis, etc., and also the expression of crucial growth and differentiation associated genes such as growth factors, growth factor receptors, bone morphogenic proteins. etc. for repairing the damages acquired during aging and degenerative conditions; f) BSP promoter driven therapy can be modulated by a variety of compounds to produce an enhanced desired effect for the specific application intended; these compounds include, but are not limited to, vitamin C or D, glucocorticoid, TGF-β, PTH/PTHrp, etc.

Prostate cancer metastasizes primarily to the skeleton. The present invention demonstrates that BSP is expressed highly in a wide variety of prostate cancer cells, and that the BSP promoter can efficiently drive expression of chosen reporter gene and thus also drive a toxic and/or therapeutic gene when in conjunction with this promoter, and a gene delivery vehicle. This expression includes both osteoblastic prostate cancer cells and osteolytic prostate cancer cells, and also androgen-dependent and androgen-independent prostate cancer types. Similarly, the present invention will also be useful in other tumors, including, but not limited to, osteosarcoma, colon, lung, breast, and brain tumors. Furthermore, the BSP promoter has various activation elements embedded within its DNA base pairs and the present invention shows that concurrent administration of glucocorticoid can effect increasing expression of target genes in certain tumor types (C4-2B4). Thus, in certain patients with similar tumor phenotypes, an augmentation of therapeutic effects would be elicited in vivo when glucocorticoid is administered simultaneously with BSP promoter-driven therapy. Also, the present invention demonstrates that L-ascorbic acid enhances the effect of BSP promoter-driven activity in certain osteotropic tumor phenotypes. In general, it is realized that the osteoblastic and mineralization phenotype is augmented in cells expressing bone noncollagenous proteins (such as BSP) in the presence of L-ascorbic acid (vitamin C), and thus, once again the therapeutic benefit of this BSP promoter-driven gene therapy can be augmented by concurrent administration of L-ascorbic acid in certain patients in vivo whose osteotropic tumors contain this or a similar acting phenotype. Indeed, it is fully appreciated that by manipulating the various hormone/compound and/or vitamin responsive elements (e.g. glucocorticoid, vitamin C or D, transforming growth factor beta (TGF-P), AP-1, parathyroid hormone (PTH/PTHrp), etc.) either already in the BSP promoter sequences, or by genetically engineering them into the native BSP promoter, that this unique and novel invention can be readily further regulated to provide optimal therapeutic effects for a variety of clinical applications.

Thus, a novel therapeutic agent comprising a BSP promoter linked with an appropriate delivery vector and therapeutic (or toxic) gene is conceived to be generated, including, but not limited to, Ad-BSP-TK. This recombinant, novel system will efficiently express therapeutic action and selectively target and induce the killing of osteoblast lineage cells and a wide spectrum of tumors or other susceptible benign tissues that have acquired the potential ability to calcify.

Further, a new recombinant therapy agent, such as, but not limited to AdBSP-TK, will be available to patients afflicted not only with osteosarcoma or prostate cancer, but also lung, breast, thyroid, myeloma, melanoma, colon, brain and other calcifying tumors, or susceptible benign tissues.

Other toxins or therapeutic genes may be used with the BSP promoter-driven therapy instead of the mentioned Tk. These include genes for cytosine deaminase, tumor suppresser genes, cyclic regulatory proteins, including various cytokines, growth or differentiation factors and others all can be ligated to BSP promoter in place of the mentioned Tk gene. In addition, other vector delivery systems can be generated and combined with the BSP promoter to effectively elicit the desired therapeutic response.

### 11 EXAMPLE

### Construction and Production of the Replication-Competent AdBSP-E1A

All plasmids are constructed according to standard published protocols (Bett, A. J., Haddara, W., Prevec, L. and Graham, F. L. An efficient and flexible system for construction of adenovirus vectors with insertions or deletions in early regions 1 and 3. Proc. Natl. Acad. Sci. U S A., 91:8802, 1994). Briefly, a Bam HI-Xca I fragment containing the backbone of an Ad5 vector from bp 549 to bp 5792 is digested from pXC 548C, a derivative of plasmid pXCl (McKinnon, R. D., Bacchetti, S. and Graham, F. L.

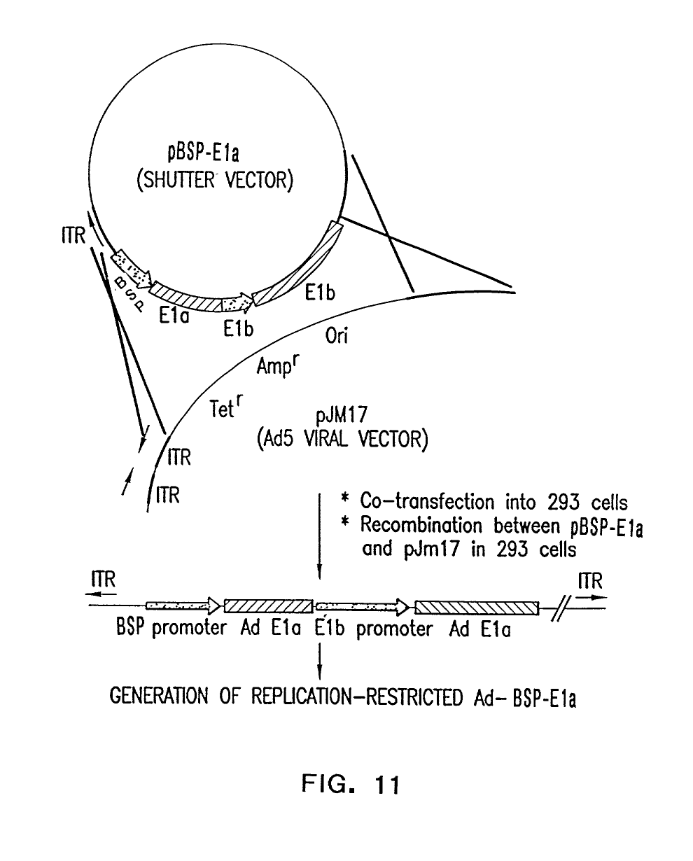

Tn5 mutagenesis of the transforming genes of human adenovirus type 5. Gene, 19:33, 1982), and inserted into pE1sp1B (obtainable from Dr. Frank Graham, MacMaster University, Hamilton, Ontario, Canada) between Bam HI and Xca I site to create a pBPAEII shuttle vector. A pBSPE1a shuttle vector is constructed by inserting a 1418 bp fragment of human BSP promoter which is cut from pII1.5 TK using Xho I and Sal I Enzymes into the Xho I site of pBPAEII to drive the Ad5 E1a gene. The shuttle pBSPE1a vector is co-transfected with a replication-defective recombinant Ad vector, pJM17, into 293 cells by the N-[1-(2,3-dioleoyloxyl)propyl]-N,N,N-trimethylammoniummethyl sulfate (Boehringer Mannheim Biochemicals)-mediated transfection method (Zhang, W-W., Fang, X., Branch, C. D., Mazur, W., French, B. A. and Roth, J. A. Generation and identification of recombinant adenovirus by liposome-mediated transfection and PCR analysis. Biotechniques, 15:868, 1993) to generate a partially E3-deleted replication-competent adenovirus, Ad-BSP-E1a, as depicted in FIG. 11. The resulting Ad-BSP-E1a is demonstrated to replicate in a restricted manner only in cells that expressed BSP promoter activity. The culture medium of the 293 cells showing complete cytopathic effect is collected and centrifuged at 1,000×g for 10 min. The pooled supernatants are aliquoted and stored at −80 C as primary viral stock. Viral stocks are propagated in 293 cells, and selected clones of Ad-BSP-E1a virus are obtained by plaque purification according to the method of Graham and Pervec (Graham, F. L. and Prevec, L. Manipulation of adenovirus vectors. Vol. 7, pp. 109-128. Clifton, N. J.: The Humana Press, Inc., 1991). Viral clones may be selected, propagated in 293 cells, and harvested 36 to 40 h after infection, pelleted, resuspended in PBS, and lysed. Cell debris is removed by subjecting the cells to centrifugation, and the virus in the cell lysate is then purified by CsCl₂ gradient centrifugation. Concentrated virus is dialyzed, aliquoted, and stored at −80 C. The viral titer may be determined by plaque assay as described previously (Gotoh, A., Ko, S. C., Shirakawa, T., Cheon, J., Kao, C., Miyamoto, T., Gardner, T. A., Ho, L. J., Cleutjens, C. B., Trapman, J., Graham, F. L. and Chung, L. W. K. Development of prostate-specific antigen promoter-based gene therapy for androgen-independent human prostate cancer. J. Urol., 160:220, 1998).

The invention described and claimed herein is not to be limited in scope by the specific embodiments herein disclosed since these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

All publications, patents and patent applications mentioned in this specification are herein incorporated by reference to the same extent as if each individual publication, patent or patent application was specifically and individually indicated to be incorporated by reference.

What is claimed is:

1. A therapeutic agent comprising a bone sialoprotein (“BSP”) promoter, a delivery vector and a toxic, therapeutic and/or heterologous coding sequence.

2. The therapeutic agent of claim 1, further comprising a prodrug.

3. The therapeutic agent of claim 2, wherein said prodrug is selected from the group consisting of acyclovir (“ACV”) and gancyclovir (“GCV”).

4. The therapeutic agent of claim 1, further comprising a glucocorticoid or an L-ascorbic acid.

5. The therapeutic agent of claim 1, wherein said BSP promoter comprises nucleotides −1349 to +69 depicted in FIG. 8.

6. The therapeutic agent of claim 1, wherein said delivery vector comprises a viral vector.

7. The therapeutic agent of claim 6, wherein said viral vector is an adenovirus.

8. The therapeutic agent of claim 1, wherein said delivery vector comprises a liposome.

9. The therapeutic agent of claim 1, wherein said toxic coding sequence is selected from the group consisting of thymidine kinase and cytosine deaminase.

10. The therapeutic agent of claim 1, wherein said therapeutic coding sequence is selected from the group consisting of growth factors, cytokines, therapeutic proteins, hormones and peptide fragments of hormones, inhibitors of cytokines, peptide growth and differentiation factors, interleukins, chemokines, interferons, colony stimulating factors and angiogenic factors.

11. The therapeutic agent of claim 1, wherein said heterologous coding sequence is a reporter gene.

12. The therapeutic agent of claim 11, wherein said reporter gene is a luciferase.

13. A method for identifying a test compound capable of modulating osteotropic-specific gene expression comprising:
(a) measuring the level of expression of a reporter gene under the control of a BSP regulatory region, or a transcriptionally active fragment thereof, in the presence and absence of said test compound, such that if the level obtained in the presence of the test compound differs from that obtained in its absence, then a compound which modulates osteotropic-specific gene expression is identified.

14. The method of claim 13 wherein the reporter gene is luciferase.

15. A pharmaceutical composition comprising the test compound identified by the method in claim 13.

16. A method for delivery of a toxic and/or therapeutic molecule comprising, introducing into osteotropic cells of a subject a vector comprising a BSP regulatory region sequence, or transcriptionally active fragment thereof, operatively linked to a heterologous nucleic acid which encodes said toxic and/or therapeutic molecule.

17. A method for treating and/or ameliorating an osteotropic-related disease or disorder comprising introducing into osteotropic cells of a subject a vector comprising a BSP regulatory region sequence, or transcriptionally active fragment thereof, operatively linked to a heterologous nucleic acid whose gene product is capable of treating and/or ameliorating said disease or disorder.

18. A method for treating and/or ameliorating an osteotropic-related cancer or other proliferative disorder comprising introducing into a cell of said cancer or other proliferative disorder of a subject a vector comprising a BSP regulatory region sequence, or transcriptionally active fragment thereof, a delivery vector and a toxic, therapeutic and/or heterologous coding sequence whose gene product is capable of killing said cell.

19. The method of claim 18 wherein said cancer or other proliferative disorder is selected from the group consisting of osteosarcoma, prostate, breast, colon, lung, brain, multiple myeloma, thyroid, melanoma or any other disease or disorder with calcification potential.

20. The method of claim 18 further comprising introducing a prodrug.

21. The method of claim 20 wherein said prodrug is selected from the group consisting of ACV and GCV.

22. The method of claim 20 wherein said introducing comprises administration via direct application, or systemic application via intravenous administration, intra-arterial administration, intra-tumoral administration, perfusion and oral administration.

23. The method of claim 18, wherein said BSP regulatory region sequence comprises nucleotides −1349 to +69 depicted in FIG. 8.

24. The method of claim 23, wherein said BSP regulatory region sequence comprises a nucleotide sequence which hybridizes under highly stringent conditions to the complement of nucleotides −1349 to +69 depicted in FIG. 8.

25. The method of claim 23, wherein said BSP regulatory region sequence comprises a nucleotide sequence which hybridizes under moderately stringent conditions to the complement of nucleotides −1349 to +69 depicted in FIG. 8.

26. A method for preventing or delaying an osteotropic-related disorder comprising introducing into osteotropic cells of a subject a vector comprising a BSP regulatory region sequence, or transcriptionally active fragment thereof, operatively linked to a heterologous nucleic acid which encodes a therapeutic molecule which is capable of preventing or delaying said disorder.

27. A method for promoting bone repair comprising administering a polynucleotide to an area where bone repair is necessary, wherein said polynucleotide comprises a BSP regulatory region sequence, or transcriptionally active fragment thereof, a delivery vector and therapeutic coding sequence whose gene product is capable of promoting said bone repair.

28. The method of claim 27, wherein said therapeutic coding sequence is selected from the group consisting of growth factors, cytokines, therapeutic proteins, hormones and peptide fragments of hormones, inhibitors of cytokines, peptide growth and differentiation factors, interleukins, chemokines, interferons, colony stimulating factors and angiogenic factors.

29. A method for modulating immune functions comprising administering a polynucleotide to an area where modulation of immune function is necessary, wherein said polynucleotide comprises a BSP regulatory region sequence, or transcriptionally active fragment thereof, a delivery vector and therapeutic coding sequence whose gene product is capable of modulating immune functions.

30. The method of claim 29, wherein said therapeutic coding sequence is selected from the group consisting of interferons alpha, beta or gamma; tumour necrosis factor; granulocyte-macrophage colony-stimulating factor (GM-CSF), granulocyte colony-stimulating factor (G-CSF), macrophage colony-stimulating factor (N-CSF), chemokines such as neutrophil activating protein NAP, macrophage chemoattractant and activating factor MCAF, RANTES, macrophage inflammatory peptides MIP-1a and MIP-1b, complement components and their receptors, accessory molecules such as 87.1, 87.2, ICAM-1.2 or 3 or cytokine receptors.
